(19) United States
(12) Patent Application Publication
(10) Pub. No.: US 2011/0195930 A1
(43) Pub. Date: Aug. 11, 2011

(54) PYRIDINE DERIVATIVE OR ITS SALT, PESTICIDE CONTAINING IT AND PROCESS FOR ITS PRODUCTION

(75) Inventors: Takahiro Haga, Kusatsu-shi (JP); Masayuki Morita, Osaka (JP); Kazuhisa Kiriyama, Shiga (JP); Kumiko Azuma, Shiga (JP)

(73) Assignee: Ishihara Sangyo Kaisha, Ltd., Osaka-shi (JP)

(21) Appl. No.: 13/125,582

(22) PCT Filed: Nov. 6, 2009
(86) PCT No.: PCT/JP09/69305

§ 371 Date: Apr. 22, 2011

(30) Foreign Application Priority Data
Nov. 17, 2008 (JP) 2008-292881

(51) Int. Cl. A01N 55/10; C07D 401/06; A01N 43/40; C07F 7/18; C07D 401/14; A01P 7/00

(52) U.S. Cl. 514/63; 546/272.7; 514/341; 546/272.4; 514/340; 546/14; 546/256; 514/333

(57) ABSTRACT

To provide a novel pesticide. The present invention provides a pesticide which contains, as an active ingredient, novel pyridine derivative represented by the formula (I) or its salt: wherein R¹ is alkyl, cycloalkyl, alkoxyalkyl or OR³; R² is 1H-1,2,4-triazol-1-yl which may be substituted, 1H-imidazol-1-yl which may be substituted, 1H-1,2,3-triazol-1-yl which may be substituted, or 4H-1,2,4-triazol-4-yl which may be substituted; X is alkyl which may be substituted, cycloalkyl which may be substituted, halogen, nitro, etc.; R³ is alkyl which may be substituted, cycloalkyl which may be substituted, alkenyl which may be substituted, alkynyl which may be substituted, etc.; m is an integer of from 1 to 4.

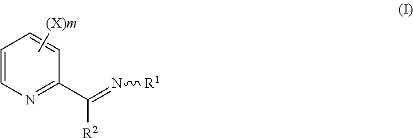

### TECHNICAL FIELD

The present invention relates to a pesticide containing a novel pyridine derivative or its salt as an active ingredient.

### BACKGROUND ART

Patent Document 1 discloses that oxime derivatives having a specific chemical structure are useful as insecticides. However, it discloses nothing specific about the compounds of the present invention represented by the formula (I) given hereinafter.

Patent Document 1: JP-A-03-68559

### DISCLOSURE OF THE INVENTION

### Object to be Accomplished by the Invention

For many years, many pesticides have been used, but many of them have various problems such that the effects are inadequate, their use is restricted as pests have acquired resistance, etc. Accordingly, it is desired to develop a novel pesticide substantially free from such problems, for example, a pesticide capable of controlling various pests which create problems in agricultural and horticultural fields or a pesticide capable of controlling pests parasitic on animals.

### Means to Accomplish the Object

The present inventors have conducted various studies on pyridine derivatives in an effort to find a superior pesticide. As a result, they have found that a novel pyridine derivative represented by the formula (I) given hereinafter has a high pesticidal effect against pests at a low dose, and have accomplished the present invention.

Namely, the present invention relates to a pyridine derivative represented by the formula (I) or its salt:

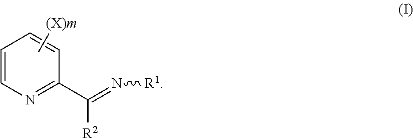

wherein R¹ is alkyl, cycloalkyl, alkoxyalkyl or OR³; R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, phenoxy which may be substituted by B, hydroxyl, NR⁴R⁵, OCOR⁶, OCOOR⁶, OS(O)ₙR⁶, aryl which may be substituted by B, heteroaryl which may be substituted by B, COR⁶, COOR⁶, S(O)ₙR⁶or CONR⁴R⁵; R³ is alkyl which may be substituted by D, cycloalkyl which may be substituted by E, alkenyl which may be substituted by D, alkynyl which may be substituted by D, phenylalkyl which may be substituted by E, pyridylalkyl which may be substituted by E, phenyl which may be substituted by E, silyl which is substituted by E, N-alkylcarbamoyl, N-alkoxycarbamoyl or N,N-dialkylcarbamoyl; R⁴ is a hydrogen atom or alkyl; R⁵ is a hydrogen atom, alkyl which may be substituted by A, cycloalkyl which may be substituted by B, arylalkyl which may be substituted by B, heteroarylalkyl which may be substituted by B, COR⁶, COOR⁶, S(O)ₙR⁶ or CH₂CN; R⁶ is alkyl, haloalkyl, or aryl which may be substituted by B; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; D is at least one substituent selected from the group consisting of cycloalkyl, halogen, alkoxy, haloalkoxy, alkylthio, cyano, nitro, alkylcarbonyl, haloalkylcarbonyl, alkoxycarbonyl and alkylsilyl; E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl, alkylsilyl, tetrahydropyranyl, 1,3-dioxolan-2-yl and N,N-dialkylamino; m is an integer of from 1 to 4; and n is 1 or 2.

The present invention further relates to a pesticide containing the pyridine derivative of the formula (I) or its salt *as an active ingredient, a method for controlling a pest by applying it, and a process for its production.

### Effects of the Invention

A pesticide containing the pyridine derivative of the above formula (I) or its salt as an active ingredient has a high pesticidal effect against pests at a low dose.

### BEST MODE FOR CARRYING OUT THE INVENTION

When m in the formula (I) is an integer of from 2 to 4, the respective X′s may be the same or different.

As the halogen in the formula (I), an atom of fluorine, chlorine, bromine or iodine may be mentioned. The number of halogens as the substituents may be 1 or more, and if more, the respective halogens may be the same or different. Further, the positions for substitution of such halogens may be any positions.

The alkyl in the formula (I) may be linear or branched. As its specific example, C₁₋₆ alkyl such as methyl, ethyl, propyl, isopropyl, butyl, tert-butyl, pentyl or hexyl may be mentioned.

As the cycloalkyl in the formula (I), C₃₋₆ cycloalkyl such as cyclopropyl, cyclobutyl, cyclopentyl or cyclohexyl may, for example, be mentioned.

The alkenyl in the formula (I) may be linear or branched. As its specific example, C₂₋₆ alkenyl such as vinyl, 1-propenyl, allyl, isopropenyl, 1-butenyl, 1,3-butadienyl or 1-hexenyl may be mentioned.

The alkynyl in the formula (I) may be linear or branched. As its specific example, C₂₋₆ alkynyl such as ethynyl, 2-butynyl, 2-pentynyl, 3-methyl-1-butynyl, 2-penten-4-ynyl or 3-hexynyl may be mentioned.

As the aryl in the formula (I), C₆₋₁₀ aryl such as phenyl or naphthyl may, for example, be mentioned.

The heteroaryl in the formula (I) may be monocyclic heteroaryl or fused heteroaryl, and it may contain from 1 to 4 atoms of at least one type selected from the group consisting of O, S and N. Its specific example may, for example, be 5-membered heteroaryl such as furyl, thienyl, pyrrolyl, oxazolyl, isoxazolyl, thiazolyl, isothiazolyl, imidazolyl, pyrazolyl, triazolyl, oxadiazolyl, thiadiazolyl or tetrazolyl; 6-membered heteroaryl such as pyridyl, thiazinyl, pyridazinyl, pyrimidinyl, pyrazinyl or triazinyl; or 8- to 10-membered fused heteroaryl such as benzofuranyl, isobenzofuranyl, benzothienyl, isobenzothienyl, indolyl, isoindolyl, benzoxazolyl, benzothiazolyl, indazolyl, benzimidazolyl, quinolyl, isoquinolyl, phthalazinyl, quinazolinyl, quinoxalinyl, imidazopyridyl, naphthyridinyl or pteridinyl.

The salt of the pyridine derivative of the above formula (I) includes all kinds so long as they are acceptable in this technical field. For example, an inorganic acid salt such as a hydrochloride, a perchlorate, a sulfate or a nitrate, or an organic acid salt such as an acetate or a methanesulfonate, may be mentioned.

The pyridine derivative of the above formula (I) may have isomers such as optical isomers or geometrical isomers, and such isomers and mixtures thereof are both included in the present invention. Further, in the present invention, various isomers other than those mentioned above, may be included within the scope of the common knowledge in this technical field.

The pyridine derivative of the above formula (I) or its salt can be produced by the following production processes [1], [2], [3] and [4] and in accordance with a usual method for producing a salt.

Now, the respective production processes will be described in detail with reference to the reaction flowcharts.

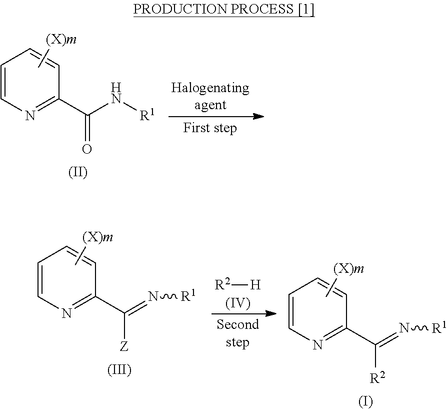

In production process [1], Z is halogen, and R¹, R², X and m are as defined above. As the halogen for Z, an atom of fluorine, chlorine, bromine or iodine may be mentioned.

In the first step of the production process [1], a compound of the formula (II) is reacted with a halogenating agent to produce a compound of the formula (III). The halogenating agent may, for example, be phosphorus pentachioride; phosphorus oxychloride; thionyl chloride; triphenylphosphine and carbon tetrachloride; or triphenylphosphine and carbon tetrabromide. The halogenating agent may be used in a proportion of from 1 to 5 equivalents, preferably from 1 to 2 equivalents, per mol of the compound of the formula (II). This reaction may be carried out in the presence of a solvent, as the case requires. The solvent is not particularly limited so long as it presents no adverse effect to the reaction, and it may, for example, be a halogenated hydrocarbon such as chloroform, dichloromethane, carbon tetrachloride, carbon tetrabromide or 1,2-dichloroethane; an aromatic hydrocarbon such as benzene, toluene or xylene; or a nitrile such as acetonitrile or propiononitrile. The reaction temperature is usually from 0 to 150° C., preferably from 50 to 120° C. The reaction time is usually from 1 to 24 hours. The compound of the formula (III) produced by this reaction step can be used in the second step of the production process [1] without being isolated.

In the second step of the production process [1], the compound of the formula (III) is reacted with a compound of the formula (IV) to produce a compound of the formula (I). The compound of the formula (IV) can be used in a proportion of from 1 to 5 equivalents, preferably from 1 to 2 equivalents, per mol of the compound of the formula (III). This reaction may be carried out in the presence of a base as the case requires. The base may, for example, be an alkali metal hydride such as sodium hydride or potassium hydride; an alkali metal hydroxide such as sodium hydroxide or potassium hydroxide; an alkali metal alkoxide such as sodium methoxide, sodium ethoxide or potassium tertiary butoxide; an alkali metal carbonate such as sodium carbonate or potassium carbonate; an alkali metal hydrogencarbonate such as sodium hydrogencarbonate or potassium hydrogencarbonate; or an organic base such as triethylamine or pyridine. The base may be used in a proportion of from 0.01 to 3 equivalents, preferably from 1 to 2 equivalents, per mol of the compound of the formula (III). This reaction can be carried out usually in the presence of a solvent. The solvent is not particularly limited so long as it presents no adverse effect to the reaction, and it may, for example, be an ether such as diethyl ether, dipropyl ether, dibutyl ether, tetrahydrofuran or dioxane; an ester such as methyl acetate or ethyl acetate; a nitrite such as acetonitrile or propiononitrile; an acid amide such as N,N-dimethylformamide, N,N-dimethylacetamide or N-methylpyrrolidinone; or a solvent mixture thereof. The reaction temperature is usually from 0 to 120° C., preferably from 20 to 100° C. The reaction time is usually from 1 to 24 hours.

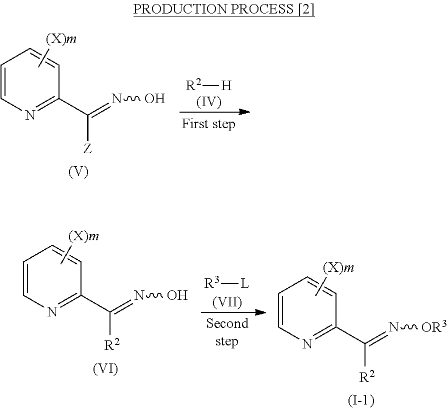

In production process [2], L is a leaving group, and R², R³, X, Z and m are as defined above. The leaving group for L may, for example, be halogen, alkylsulfonyloxy, trifluoromethanesulfonyloxy, or benzenesulfonyloxy which may be substituted by alkyl.

In the first step of the production process [2], a compound of the formula (V) is reacted with a compound of the formula (IV) to produce a compound of the formula (VI). The compound of the formula (IV) may be used in a proportion of from 1 to 5 equivalents, preferably from 1.1 to 3 equivalents, per mol of the compound of the formula (V). This reaction may usually be carried out in the presence of a base and a solvent. As the base, the same one as mentioned for the second step of the above production process [1] may be mentioned. The base may be used in a proportion of from 1 to 5 equivalents, preferably from 1 to 3 equivalents, per mol of the compound of the formula (V). The solvent is not particularly limited so long as it presents no adverse effect to the reaction, and for example, it may be the same one as mentioned in the second step of the above production process [1]. The reaction temperature is usually from −20 to 100° C., preferably from −10 to 50° C. The reaction time is usually from 0.5 to 5 hours.

In the second step of the production process [2], the compound of the formula (VI) is reacted with a compound of the formula (VII) to produce a compound of the formula (I-1). The compound of the formula (VII) may be used in a proportion of from 1 to 5 equivalents, preferably from 1.2 to 3 equivalents, per mol of the compound of the formula (VI). This reaction may be carried out in the presence of a base, as the case requires. The base may, for example, be an alkali metal hydride such as sodium hydride or potassium hydride; an alkali metal alkoxide such as sodium methoxide, sodium ethoxide or potassium tertiary butoxide; an alkali metal carbonate such as sodium carbonate or potassium carbonate; or an alkali metal hydrogencarbonate such as sodium hydrogencarbonate or potassium hydrogencarbonate. The base may be used in a proportion of from 0.8 to 3 equivalents, preferably from 1 to 2 equivalents, per mol of the compound of the formula (VI). This reaction may usually be carried out in the presence of a solvent. The solvent is not particularly limited so long as it presents no adverse effect to the reaction, and it may, for example, be the same one as mentioned in the second step of the above production process [1]. The reaction temperature is usually from 0 to 100° C., preferably from 10 to 50° C. The reaction time is usually from 1 to 5 hours.

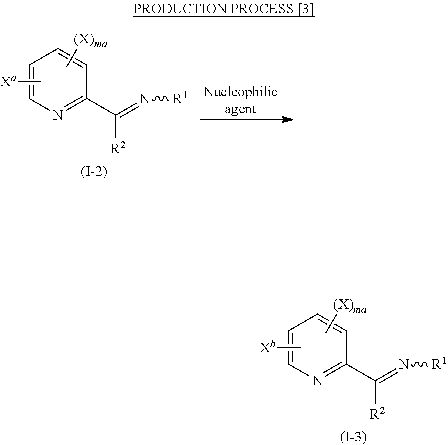

In the production process [3], X^{a} is a leaving group; X^{b} is halogen, cyano, alkoxy which may be substituted by A, cycloalkyloxy which may be substituted by B, arylalkoxy which may be substituted by B, silylalkoxy which is substituted by B, alkylthio which may be substituted by A, alkenyloxy which may be substituted by A, alkynyloxy which may be substituted by A, phenoxy which may be substituted by B, NR⁴R⁵, OCOR⁶, OCOOR⁶ or OS(O)ₙR⁶; ma is an integer of from 0 to 3; and R¹, R², X, R⁴, R⁵, R⁶, A, B and n are as defined above. The leaving for X^{a} may, for example, be halogen, alkylsulfonyloxy, trifluoromethanesulfonyloxy, or benzenesulfonyloxy which may be substituted by alkyl.

In the production process [3], a compound of the formula (I-2) is reacted with a nucleophilic agent to produce a compound of the formula (I-3). The nucleophilic agent may, for example, be a metal halide such as cesium fluoride, potassium fluoride or potassium iodide; an alkali metal cyanide such as sodium cyanide or potassium cyanide; an alkali metal alkoxide such as sodium methoxide or sodium ethoxide; an alkali metal thiolate such as sodium thiomethoxide; or an amine represented by the formula HNR⁴R⁵ (wherein R⁴ and R⁵ are as defined above). The nucleophilic agent may be used in a proportion of from 1 to 10 equivalents, preferably from 1 to 3 equivalents, per mol of the compound of the formula (I-2). This reaction may be carried out in the presence of a base, as the case requires. The base may, for example, be the same one as mentioned in the second step of the above production process [1]. The base may be used in a proportion of from 1 to 5 equivalents, preferably from 1 to 3 equivalents, per mol of the compound of the formula (I-2).

This reaction may usually be carried out in the presence of a solvent. The solvent is not particularly limited so long as it presents no adverse effect to the reaction, and it may, for example, be an alcohol such as methanol, ethanol, propanol or butanol; an aromatic hydrocarbon such as benzene, toluene or xylene; an aliphatic hydrocarbon such as pentane, hexane, heptane, petroleum ether, ligroin or petroleum benzine; an ether such as diethyl ether, butyl ethyl ether, tetrahydrofuran, dioxane or dimethoxyethane; an ester such as methyl acetate or ethyl acetate; a halogenated hydrocarbon such as chlorobenzene, chloroform, dichloromethane, carbon tetrachloride or 1,2-dichloroethane; a nitrile such as acetonitrile or propiononitrile; an acid amide such as N,N-dimethylformamide, N,N-dimethylacetamide or N-methylpyrrolidinone; a sulfoxide such as dimethylsulfoxide; or a solvent mixture thereof. The reaction temperature is usually from −100° C. to the reflux temperature of the reaction mixture, preferably from −30° C. to 150° C. The reaction time is usually from about 1 minute to 96 hours.

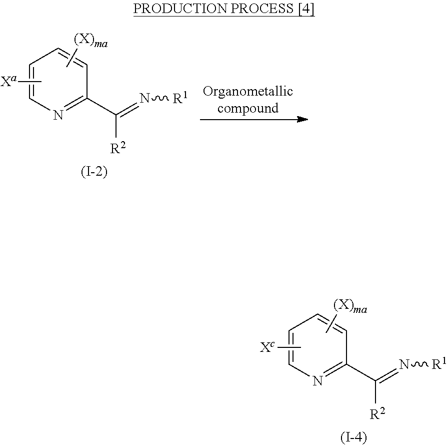

In the production process [4], X^{c} is alkyl which may be substituted by A, cycloalkyl which may be substituted by B, alkenyl which may be substituted by A, alkynyl which may be substituted by A, aryl which may be substituted by B, or heteroaryl which may be substituted by B; and R¹, R², X, X^{a}, A, B and ma are as defined above.

In the production process [4], a compound of the formula (I-2) is reacted with an organometallic compound to produce a compound of the formula (I-4). The organometallic compound may, for example, be an organocopper compound, an organoboron compound, an organozinc compound, an organomagnesium compound, an organolithium compound, an organotin compound or an organosilicon compound. The organometallic compound may be used in a proportion of from 1 to 5 equivalents, preferably from 1 to 3 equivalents, per mol of the compound of the formula (I-2). This reaction may usually be carried out in the presence of a catalyst and a base. The catalyst may, for example, be a palladium compound or a nickel compound. The catalyst may be used in a proportion of from 0.0001 to 0.2 equivalent, preferably from 0.001 to 0.1 equivalent, per mol of the compound of the formula (I-2). The base may be the same one as mentioned in the second step of the above-mentioned production process [1]. The base may be used in a proportion of from 1 to 10 equivalents, preferably from 1 to 5 equivalents, per mol of the compound of the formula (I-2).

This reaction may usually be carried out in the presence of a solvent. The solvent is not particularly limited so long as it presents no adverse effect to the reaction, and it may, for example, be water; an aromatic hydrocarbon such as benzene, toluene or xylene; an aliphatic hydrocarbon such as pentane, hexane, heptane, petroleum ether, ligroin or petroleum benzine; an ether such as diethyl ether, dipropyl ether, dibutyl ether, tetrahydrofuran or dioxane; a ketone such as acetone, methyl ethyl ketone, dimethyl ketone, diethyl ketone or methyl isobutyl ketone; an ester such as methyl acetate or ethyl acetate; a halogenated hydrocarbon such as chloroform, dichloromethane, carbon tetrachloride or 1,2-dichloroethane; a nitrile such as acetonitrile or propiononitrile; an amide such as N,N-dimethylformamide, N,N-dimethylacetamide or N-methylpyrrolidinone; a sulfoxide such as dimethylsulfoxide; a sulfone such as sulfolane; a phosphoric acid amide such as hexamethylphosphoramide; or a solvent mixture thereof. The reaction temperature is usually from −100° C. to the reflux temperature of the reaction mixture, preferably from −30° C. to 150° C. The reaction time is usually from about 1 minute to 96 hours.

The compound of the formula (II) to be used in the first step of the production process [1] may be produced, for example, by the following production process [A] or [B]. Now, the respective production processes will be described in detail with reference to the reaction flowcharts.

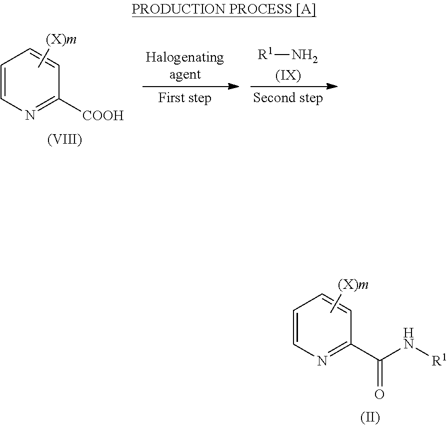

In production process [A], R¹, X and m are as defined above.

The production process [A] comprises the above first step and second step, and a compound of the formula (II) can be produced from the compound of the formula (VIII). The product of the first step may be used in the second step without being isolated.

In the first step of production process [A], a compound of the formula (VIII) is reacted with a halogenating agent. The halogenating agent may, for example, be thionyl chloride or oxalyl dichloride. The halogenating agent may be used in a proportion of from 1 to 10 equivalents, preferably from 1 to 5 equivalents, per mol of the compound of the formula (VIII). This reaction may be carried out in the presence of a reaction accelerator, as the case requires. The reaction accelerator may, for example, be N,N-dimethylformamide or a base. The base may, for example, be an organic base such as triethylamine, pyridine or 4-dimethylaminopyridine. The reaction accelerator may be used in a proportion of from 0.001 to 3.0 equivalents, preferably from 0.01 to 0.5 equivalent, per mol of the compound of the formula (VIII). This reaction may be carried out in the presence of a solvent, as the case requires. The solvent is not particularly limited so long as it presents no adverse effect to the reaction, and it may, for example, be an aromatic hydrocarbon such as benzene, toluene or xylene; an aliphatic hydrocarbon such as pentane, hexane, heptane, petroleum ether, ligroin or petroleum benzine; an ether such as diethyl ether, dipropyl ether, dibutyl ether, tetrahydrofuran or dioxane; an ester such as methyl acetate or ethyl acetate; a halogenated hydrocarbon such as chloroform, dichloromethane, carbon tetrachloride or 1,2-dichloroethane; or a solvent mixture thereof. Further, a halogenating agent such as thionyl chloride or oxalyl dichloride may be used as a solvent. The reaction temperature is usually from 0 to 150° C., preferably from 50 to 100° C. The reaction time is usually from 0.5 to 6 hours.

In the second step of the production process [A], the product in the first step of the production process [A] is reacted with a compound of the formula (IX) or its salt to obtain the compound of the formula (II). The compound of the formula (IX) may be used in a proportion of from 1 to 10 equivalents, preferably from 1 to 5 equivalents, per mol of the compound of the formula (VIII). This reaction may be carried out in the presence of a base, as the case requires. The base may, for example, be an organic base such as triethylamine, pyridine or 4-dimethylaminopyridine. The base may be used in a proportion of from 0.05 to 10 equivalents, preferably from 0.1 to 2.5 equivalents, per mol of the compound of the formula (VIII). This reaction may usually be carried out in the presence of a solvent. The solvent is not particularly limited so long as it presents no adverse effect to the reaction, and it may, for example, be an aromatic hydrocarbon such as benzene, toluene or xylene; an aliphatic hydrocarbon such as pentane, hexane, heptane, petroleum ether, ligroin or petroleum benzine; an ether such as diethyl ether, dipropyl ether, dibutyl ether, tetrahydrofuran or dioxane; an ester such as methyl acetate or ethyl acetate; a halogenated hydrocarbon such as chloroform, dichloromethane, carbon tetrachloride or 1,2-dichloroethane; an acid amide such as N,N-dimethylformamide, N,N-dimethylacetamide or N-methylpyrrolidinone; or a solvent mixture thereof. The reaction temperature is usually from −10 to 100° C., preferably from 0 to 30° C. The reaction time is usually from 0.5 to 6 hours.

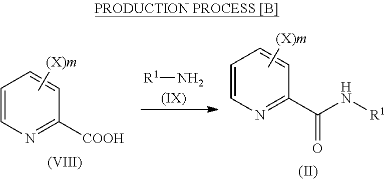

In production process [B], R¹, X and m are as defined above.

In the production process [B], the compound of the formula (VIII) is reacted with a compound of the formula (IX) in the presence of a condensation agent to produce the compound of the formula (II). The compound of the formula (IX) may be used in a proportion of from 1 to 10 equivalents, preferably from 2 to 5 equivalents, per mol of the compound of the formula (VIII). The condensation agent may, for example, be a carbodiimide such as dicyclohexylcarbodiimide, diisopropylcarbodiimide, N-ethyl-N′-(3-dimethylaminopropyl)carbodiimide or its salt. The condensation agent may be used in a proportion of from 1 to 5 equivalents, preferably from 1 to 2 equivalents, per mol of the compound of the formula (VIII). This reaction may be carried out in the presence of a reaction accelerator, as the case requires. The reaction accelerator may, for example, be 1-hydroxybenzotriazole, N-hydroxysuccinimide, 1-hydroxy-7-azabenzotriazole or a base. The base may, for example, be an organic base such as triethylamine, pyridine or 4-dimethylaminopyridine. The reaction accelerator may be used in a proportion of from 1 to 5 equivalents, preferably from 1 to 2 equivalents, per mol of the compound of the formula (VIII). This reaction may usually be carried out in the presence of a solvent. The solvent is not particularly limited so long as it presents no adverse effect to the reaction, and it may, for example, be an ether such as diethyl ether, dipropyl ether, dibutyl ether, tetrahydrofuran or dioxane; a halogenated hydrocarbon such as chloroform, dichloromethane, carbon tetrachloride or 1,2-dichloroethane; an acid amide such as N,N-dimethylformamide, N,N-dimethylacetamide or N-methylpyrrolidinone; or a solvent mixture thereof. The reaction temperature is usually from −10 to 100° C., preferably from 0 to 30° C. The reaction time is usually from 1 to 24 hours. The compound of the formula (V) to be used in the first step of the production process [2] may, for example, be produced by the following production process [C] or [D]. Now, the respective production processes will be described in detail with reference to the reaction flowcharts.

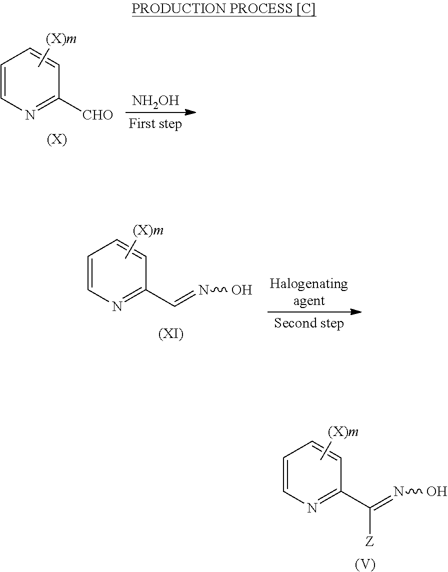

In production process [C], X, Z and m are as defined above.

In the first step of the production process [C], a compound of the formula (X) is reacted with hydroxylamine or its salt to produce a compound of the formula (XI). The hydroxylamine or its salt may be used in a proportion of from 1 to 3 equivalents, preferably from 1 to 1.5 equivalents, per mol of the compound of the formula (X). This reaction may be carried out in the presence of a base, as the case requires. The base may, for example, be an alkali metal hydroxide such as sodium hydroxide or potassium hydroxide; an alkali metal carbonate such as sodium carbonate or potassium carbonate; an alkali metal hydrogencarbonate such as sodium hydrogencarbonate or potassium hydrogencarbonate; or an organic base such as triethylamine or pyridine. The base may be used in a proportion of from 1 to 5 equivalents, preferably from 1 to 2 equivalents, per mol of the compound of the formula (X). This reaction may usually be carried out in the presence of a solvent. The solvent is not particularly limited so long as it presents no adverse effect to the reaction, and it may, for example, be water; an alcohol such as methanol, ethanol, propanol or butanol; an ether such as diethyl ether, dipropyl ether, dibutyl ether, tetrahydrofuran or dioxane; a nitrile such as acetonitrile or propiononitrile; or a solvent mixture thereof. The reaction temperature is usually from 0 to 100° C., preferably from 10 to 50° C. The reaction time is usually from 0.5 to 5 hours.

In the second step of production process [C], the compound of the formula (XI) is reacted with a halogenating agent to produce a compound of the formula (V). The halogenating agent may, for example, be N-chlorosuccinimide, N-bromosuccinimide, N-iodosuccinimide or chlorine. The halogenating agent may be used in a proportion of from 1 to 3 equivalents, preferably from 1 to 1.5 equivalents, per mol of the compound of the formula (XI). In a case where N-chlorosuccinimide is used as the halogenating agent, the reaction may be carried out in the presence of a small amount of hydrochloric acid, as the case requires. Such hydrochloric acid may be used in a proportion of e.g. from 0.01 to 0.5 equivalent, per mol of the compound of the formula (XI). This reaction may usually be carried out in the presence of a solvent. The solvent is not particularly limited so long as it presents no adverse effect to the reaction, and it may, for example, be a halogenated hydrocarbon such as chloroform, dichloromethane, carbon tetrachloride or 1,2-dichloroethane; a nitrile such as acetonitrile or propiononitrile; or an acid amide such as N,N-dimethylformamide, N,N-dimethylacetamide or N-methylpyrrolidinone. The reaction temperature is usually from 0 to 80° C., preferably from 20 to 50° C. The reaction time is usually from 0.25 to 5 hours.

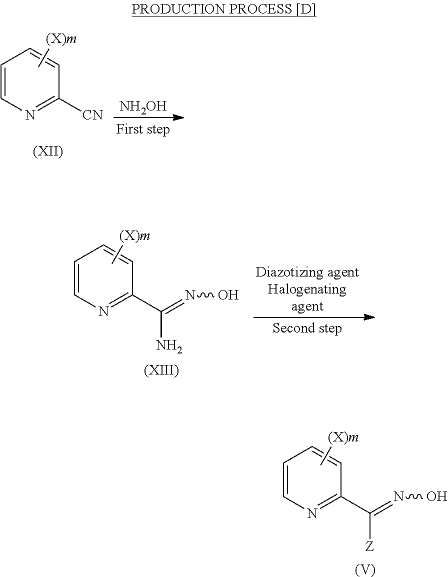

In production process [D], X, Z and m are as defined above.

In the first step of the production process [D], a compound of the formula (XII) is reacted with hydroxylamine or its salt to produce a compound of the formula (XIII). The hydroxylamine or its salt may be used in a proportion of from 1 to 3 equivalents, preferably from 1 to 1.5 equivalents, per mol of the compound of the formula (XII). This reaction may be carried out in the presence of a base, as the case requires. The base may, for example, be the same one as mentioned in the first step of the above production process [C]. The base may be used in a proportion of from 1 to 5 equivalents, preferably from 1 to 2 equivalent, per mol of the compound of the formula (XII). This reaction may usually be carried out in the presence of a solvent. The solvent may, for example, be the same one as mentioned in the first step of the above-mentioned production process [C]. The reaction temperature is usually from 0 to 100° C., preferably from 50 to 80° C. The reaction time is usually from 0.5 to 5 hours.

In the second step of the production process [D], the compound of the formula (XIII) is reacted with a diazotizing agent and a halogenating agent to produce a compound of the formula (V). The diazotizing agent may, for example, be a nitrite such as sodium nitrite; or a nitrite ester such as isoamyl nitrite. The diazotizing agent may be used in a proportion of from 1 to 3 equivalents, preferably from 1 to 1.5 equivalents, per mol of the compound of the formula (XIII). The halogenating agent may, for example, be hydrochloric acid, hydrobromic acid or copper(I) halide. The halogenating agent may be used in a proportion of from 1 equivalent to a large excess amount, per mol of the compound of the formula (XIII). This reaction may usually be carried out in the presence of a solvent. The solvent is not particularly limited so long as it presents no adverse effect to the reaction, and it may, for example, be water; an acid such as acetic acid or sulfuric acid; a nitrile such as acetonitrile or propiononitrile; or a solvent mixture thereof. Further, a halogenating agent such as hydrochloric acid or hydrobromic acid may be used as a solvent. The reaction temperature is usually from −10 to 80° C., preferably from 0 to 50° C. The reaction time is usually from 0.5 to 5 hours.

Preferred embodiments of pesticides containing the compounds of the present invention will be described below. The pesticides containing the compounds of the present invention are particularly useful, for example, as agents for controlling various pests which become problematic in the agricultural and horticultural fields, i.e. agricultural and horticultural pesticides, or as agents for controlling pests which are parasitic on animals i.e. pesticides against parasites on animals.

The agricultural and horticultural pesticides containing the compounds of the present invention are useful as an insecticide, a miticide, a nematicide or a soil pesticide, and they are effective for controlling plant parasitic mites such as two-spotted spider mite (Tetranychus urticae), carmine spider mite (Tetranychus cinnabarinus), kanzawa spider mite (Tetranychus kanzawai), citrus red mite (Panonychus citri), European red mite (Panonychus ulmi), broad mite (Polyphagotarsonemus latus), pink citrus rust mite (Aculops pelekassi) and bulb mite (Rhizoalyphus echinopus); aphids such as green peach aphid (Myzus persicae) and cotton aphid (Aphis gossypii); agricultural insect pests such as diamondback moth (Plutella xylostella), cabbage armyworm (Mamestra brassicae), common cutworm (Spodoptera litura), codling moth (cydia pomonella), bollworm (Heliothis zea), tobacco budworm (Heliothis virescens), gypsy moth (Lymantria dispar), rice leafroller (Cnaphalocrocis medinalis), smaller tea tortrix (Adoxophyes sp.), colorado potato beetle (Leptinotarsa decemlineata), cucurbit leaf beetle (Aulacophora femoralis), boll weevil (Anthonomus grandis), planthoppers, leafhoppers, scales, bugs, whiteflies, thrips, grasshoppers, anthomyiid flies, scarabs, black cutworm (Agrotis ipsilon), cutworm (Agrotis segetum) and ants; plant parasitic nematodes such as root-knot nematodes, cyst nematodes, root-lesion nematodes, white-tip nematode (Aphelenchoides besseyi), strawberry bud nematode (Nothotylenchus acris), and pine wood nematode (Bursaphelenchus xylophilus); gastropods such as slugs and snails; soil pests such as isopods such as pillbugs (Armadillidium vulgare) and pillbugs (Porcellio scaber); hygienic insect pests such as tropical rat mite (Ornithonyssus bacoti), cockroaches, housefly (Musca domestica) and house mosquito (Culex pipiens); stored grain insect such as angoumois grain moth (Sitotroga cerealella), adzuki bean weevil (Callosobruchus chinensis), red flour beetle (Tribolium castaneum) and mealworms; household goods insect pests such as casemaking clothes moth (Tinea pellionella), black carpet beetle (Attagenus japonicus) and subterranean termites; domestic mites such as mold mite (Tyrophagus putrescentiae), Dermatophagoides farinae, Chelacaropsis moorei, and so on. Among them, the agricultural and horticultural pesticides containing the compounds of the present invention are particularly effective for controlling plant parasitic mites, agricultural insect pests, plant parasitic nematodes or the like. Particularly, they are more effective for controlling plant parasitic mites and agricultural insect pests, and accordingly they are useful as an insecticide or miticide. Further, they are effective against insect pests having acquired resistance to organophosphorus, carbamate and/or synthetic pyrethroid insecticides. Moreover, the compounds of the present invention have excellent systemic properties, and by the application of the agricultural and horticultural pesticides containing the compounds of the present invention to soil treatment, not only noxious insects, noxious mites, noxious nematodes, noxious gastropods and noxious isopods in soil but also foliage pests can be controlled.

Another preferred embodiments of the pesticides containing compounds of the present invention may be agricultural and horticultural pesticides which collectively control the above-mentioned plant parasitic mites, agricultural insect pests, plant parasitic nematodes, gastropods and soil pests.

The agricultural and horticultural pesticide containing the compound of the present invention, is usually formulated by mixing the compound with various agricultural adjuvants and used in the form of a formulation such as a dust, granules, water-dispersible granules, a wettable powder, a water-based suspension concentrate, an oil-based suspension concentrate, water soluble granules, a water soluble powder, an emulsifiable concentrate, a soluble concentrate, a paste, an aerosol or an ultra low-volume formulation. However, so long as it is suitable for the purpose of the present invention, it may be formulated into any type of formulation which is commonly used in this field. Such agricultural adjuvants include solid carriers such as diatomaceous earth, slaked lime, calcium carbonate, talc, white carbon, kaoline, bentonite, kaolinite, sericite, clay, sodium carbonate, sodium bicarbonate, mirabilite, zeolite and starch; solvents such as water, toluene, xylene, solvent naphtha, dioxane, acetone, isophorone, methyl isobutyl ketone, chlorobenzene, cyclohexane, dimethylsulfoxide, N,N-dimethylformamide, N,N-dimethylacetamide, N-methyl-2-pyrrolidone, and alcohol; anionic surfactants such as a salt of fatty acid, a benzoate, an alkylsulfosuccinate, a dialkylsulfosuccinate, a polycarboxylate, a salt of alkylsulfuric acid ester, an alkyl sulfate, an alkylaryl sulfate, an alkyl diglycol ether sulfate, a salt of alcohol sulfuric acid ester, an alkyl sulfonate, an alkylaryl sulfonate, an aryl sulfonate, a lignin sulfonate, an alkyldiphenyl ether disulfonate, a polystyrene sulfonate, a salt of alkylphosphoric acid ester, an alkylaryl phosphate, a styrylaryl phosphate, a salt of polyoxyethylene alkyl ether sulfuric acid ester, a polyoxyethylene alkylaryl ether sulfate, a salt of polyoxyethylene alkylaryl ether sulfuric acid ester, a polyoxyethylene alkyl ether phosphate, a salt of polyoxyethylene alkylaryl phosphoric acid ester, and a salt of a condensate of naphthalene sulfonate with formalin; nonionic surfactants such as a sorbitan fatty acid ester, a glycerin fatty acid ester, a fatty acid polyglyceride, a fatty acid alcohol polyglycol ether, acetylene glycol, acetylene alcohol, an oxyalkylene block polymer, a polyoxyethylene alkyl ether, a polyoxyethylene alkylaryl ether, a polyoxyethylene styrylaryl ether, a polyoxyethylene glycol alkyl ether, a polyethylene glycol, a polyoxyethylene fatty acid ester, a polyoxyethylene sorbitan fatty acid ester, a polyoxyethylene glycerin fatty acid ester, a polyoxyethylene hydrogenated castor oil, and a polyoxypropylene fatty acid ester; vegetable and mineral oils such as olive oil, kapok oil, castor oil, palm oil, camellia oil, coconut oil, sesame oil, corn oil, rice bran oil, peanut oil, cottonseed oil, soybean oil, rapeseed oil, linseed oil, tung oil, and liquid paraffins; and so on. Each of the components as such adjuvants may be one or more suitably selected for use, so long as the purpose of the present invention can thereby be accomplished. Further, various additives which are commonly used, such as a filler, a thickener, an anti-settling agent, an anti-freezing agent, a dispersion stabilizer, a phytotoxicity reducing agent, an anti-mold agent, and so on, may also be employed.

The weight ratio of the compound of the present invention to the various agricultural adjuvants is usually from 0.001:99.999 to 95:5, preferably from 0.005:99.995 to 90:10.

In the actual application of such a formulation, it may be used as it is, or may be diluted to a predetermined concentration with a diluent such as water, and various spreaders e.g. surfactants, vegetable oils or mineral oils may be added thereto, as the case requires.

The application of the agricultural and horticultural pesticide containing the compound of the present invention cannot generally be defined, as it varies depending upon the weather conditions, the type of the formulation, the application season, the application site or the types or degree of outbreak of the pest insects. However, it is usually applied in a concentration of the active ingredient being from 0.05 to 800,000 ppm, preferably from 0.5 to 500,000 ppm, and the dose per unit area is such that the compound of the present invention is from 0.05 to 50,000 g, preferably from 1 to 30,000 g, per hectare. Further, the present invention includes such a method for controlling pests, particularly for controlling plant parasitic mites, agricultural insect pests or plant parasitic nematodes by such applications.

Various formulations of agricultural and horticultural pesticides containing the compounds of the present invention or their diluted compositions may be applied by conventional methods for application which are commonly employed, such as spraying (e.g. spraying, jetting, misting, atomizing, powder or grain scattering or dispersing in water), soil application (e.g. mixing or drenching), surface application (e.g. coating, powdering or covering) or impregnation to obtain poisonous feed. Further, it is possible to feed domestic animals with a food containing the above active ingredient and to control the outbreak or growth of pests, particularly insect pests, with their excrements. Furthermore, the active ingredient may also be applied by a so-called ultra low-volume application method. In this method, the composition may be composed of 100% of the active ingredient.

Further, the agricultural and horticultural pesticides containing compounds of the present invention may be mixed with or may be used in combination with other agricultural chemicals, fertilizers or phytotoxicity-reducing agents, whereby synergistic effects or activities may sometimes be obtained. Such other agricultural chemicals include, for example, a herbicide, an insecticide, a miticide, a nematicide, a soil pesticide, a fungicide, an antivirus agent, an attractant, an antibiotic, a plant hormone, a plant growth regulating agent, and so on. Especially, with a mixed pesticide having a compound of the present invention mixed with or used in combination with one or more active compounds of other agricultural chemicals, the application range, the application time, the pesticidal activities, etc. may be improved to preferred directions. The compound of the present invention and the active compounds of other agricultural chemicals may separately be formulated so that they may be mixed for use at the time of application, or they may be formulated together. The present invention includes such a mixed pesticidal composition.

The mixing ratio of the compound of the present invention to the active compounds of other agricultural chemicals can not generally be defined, since it varies depending upon the weather conditions, the types of formulations, the application time, the application site, the types or degree of outbreak of insect pests, etc., but it is usually within a range of from 1:300 to 300:1, preferably from 1:100 to 100:1, by weight. Further, the dose for the application is such that the total amount of the active compounds is from 0.1 to 50,000 g, preferably from 1 to 30,000 g, per hectare. The present invention includes a method for controlling pests by an application of such a mixed pesticide composition.

The active compounds of insect pest control agents such as insecticides, miticides, nematicides or soil pesticides in the above-mentioned other agricultural chemicals, include, for example, (by common names, some of them are still in an application stage, or test codes) organic phosphate compounds such as profenofos, dichlorvos, fenamiphos, fenitrothion, EPN, diazinon, chlorpyrifos, chlorpyrifos-methyl, acephate, prothiofos, fosthiazate, cadusafos, dislufoton, isoxathion, isofenphos, ethion, etrimfos, quinalphos, dimethylvinphos, dimethoate, sulprofos, thiometon, vamidothion, pyraclofos, pyridaphenthion, pirimiphos-methyl, propaphos, phosalone, formothion, malathion, tetrachlorvinphos, chlorfenvinphos, cyanophos, trichlorfon, methidathion, phenthoate, ESP, azinphos-methyl, fenthion, heptenophos, methoxychlor, parathion, phosphocarb, demeton-S-methyl, monocrotophos, methamidophos, imicyafos, parathion-methyl, terbufos, phosphamidon, phosmet and phorate; carbamate compounds such as carbaryl, propoxur, aldicarb, carbofuran, thiodicarb, methomyl, oxamyl, ethiofencarb, pirimicarb, fenobucarb, carbosulfan, benfuracarb, bendiocarb, furathiocarb, isoprocarb, metolcarb, xylylcarb, XMC and fenothiocarb; nereistoxin derivatives such as cartap, thiocyclam, bensultap and thiosultap-sodium; organic chlorine compounds such as dicofol, tetradifon, endosulfan, dienochlor and dieldrin; organic metal compounds such as fenbutatin oxide and cyhexatin; pyrethroid compounds such as fenvalerate, permethrin, cypermethrin, deltamethrin, cyhalothrin, tefluthrin, ethofenprox, flufenprox, cyfluthrin, fenpropathrin, flucythrinate, fluvalinate, cycloprothrin, lambda-cyhalothrin, pyrethrins, esfenvalerate, tetramethrin, resmethrin, protrifenbute, bifenthrin, zeta-cypermethrin, acrinathrin, alpha-cypermethrin, allethrin, gamma-cyhalothrin, theta-cypermethrin, tau-fluvalinate, tralomethrin, profluthrin, beta-cypermethrin, beta-cyfluthrin, metofluthrin, phenothrin and flumethrin; benzoylurea compounds such as diflubenzuron, chlorfluazuron, teflubenzuron, flufenoxuron, triflumuron, hexaflumuron, lufenuron, novaluron, noviflumuron, bistrifluron and fluazuron; juvenile hormone-like compounds such as methoprene, pyriproxyfen, fenoxycarb and diofenolan; pyrazole compounds such as fenpyroximate, fipronil, tebufenpyrad, ethiprole, tolfenpyrad, acetoprole, pyrafluprole and pyriprole; neonicotinoids such as imidacloprid, nitenpyram, acetamiprid, thiacloprid, thiamethoxam, clothianidin, dinotefuran and nithiazine; hydrazine compounds such as tebufenozide, methoxyfenozide, chromafenozide and halofenozide; pyridine compounds such as pyridalyl and flonicamid; tetronic acid compounds such as spirodiclofen; strobilurin compounds such as fluacrypyrim; pyrimidinamine compounds such as flufenerim; dinitro compounds; organic sulfur compounds; urea compounds; triazine compounds; hydrazone compounds; and other compounds such as buprofezin, hexythiazox, amitraz, chlordimeform, silafluofen, triazamate, pymetrozine, pyrimidifen, chlorfenapyr, indoxacarb, acequinocyl, etoxazole, cyromazine, 1,3-dichloropropene, diafenthiuron, benclothiaz, bifenazate, spiromesifen, spirotetramat, propargite, clofentezine, metaflumizone, flubendiamide, cyflumetofen, chlorantraniliprole, cyenopyrafen, pyrifluquinazon, fenazaquin, pyridaben, amidoflumet, chlorobenzoate, sulfiuramid, hydramethylnon, metaldehyde, HGW 86, ryanodine and verbutin. Further, microbial pesticides such as insecticidal crystal protein produced by Bacillus thuringiensis aizawai, Bacillus thuringiensis kurstaki, Bacillus thuringiensis israelensis, Bacillus thuringiensis japonensis, Bacillus thuringiensis tenebrionis or Bacillus thuringiensis, insect viruses, etomopathogenic fungi, and nematophagous fungi; antibiotics or semisynthetic antibiotics such as avermectin, emamectin-benzoate, milbemectin, milbemycin, spinosad, ivermectin, lepimectin, DE-175, abamectin, emamectin and spinetoram; natural products such as azadirachtin and rotenone; and repellents such as deet may, for example, be mentioned.

The fungicidal active compounds in the above-mentioned other agricultural chemicals include, for example, (by common names, some of them are still in an application stage, or test codes of Japan Plant Protection Association) anilinopyrimidine compounds such as mepanipyrim, pyrimethanil, cyprodinil and ferimzone; triazolopyrimidine compounds such as 5-chloro-7-(4-methylpiperidin-1-yl)-6-(2,4,6-trifluorophenyI)[1,2,4]triazolo[1,5-a]pyrimidine; pyridinamine compounds such as fluazinam; azole compounds such as triadimefon, bitertanol, triflumizole, etaconazole, propiconazole, penconazole, flusilazole, myclobutanil, cyproconazole, tebuconazole, hexaconazole, furconazole-cis, prochloraz, metconazole, epoxiconazole, tetraconazole, oxpoconazole fumarate, sipconazole, prothioconazole, triadimenol, flutriafol, difenoconazole, fluquinconazole, fenbuconazole, bromuconazole, diniconazole, tricyclazole, probenazole, simeconazole, pefurazoate, ipconazole and imibenconazole; quinoxaline compounds such as quinomethionate; dithiocarbamate compounds such as maneb, zineb, mancozeb, polycarbamate, metiram, propineb and thiram; organic chlorine compounds such as fthalide, chlorothalonil and quintozene; imidazole compounds such as benomyl, thiophanate-methyl, carbendazim, thiabendazole, fuberiazole and cyazofamid; cyanoacetamide compounds such as cymoxanil; phenylamide compounds such as metalaxyl, metalaxyl-M, mefenoxam, oxadixyl, ofurace, benalaxyl, benalaxyl-M (another name: kiralaxyl, chiralaxyl), furalaxyl and cyprofuram; sulfenic acid compounds such as dichiofluanid; copper compounds such as cupric hydroxide and oxine copper; isoxazole compounds such as hymexazol; organophosphorus compounds such as fosetyl-Al, tolciofos-methyl, edifenphos, iprobenfos, S-benzyl O,O-diisopropylphosphorothioate, O-ethyl S,S-diphenylphosphorodithioate and aluminum ethylhydrogen phosphonate; N-halogenothioalkyl compounds such as captan, captafol and folpet; dicarboximide compounds such as procymidone, iprodione and vinclozolin; benzanilide compounds such as flutolanil, mepronil, zoxamid and tiadinil; anilide compounds such as carboxin, oxycarboxin, thifluzamide, penthiopyrad, boscalid, isothianil, bixafen and mixture of 2 syn-isomers 3-(difluoromethyl)-1-methyl-N-[(1RS,4SR,9RS)-1,2,3,4-tetrahydro-9-isopropyl-1,4-methanonaphthalen-5-yl]pyrazole-4-carboxamide and 2 anti-isomers 3-(difluoromethyl)-1-methyl-N-[(1RS,4SR,9SR)-1,2,3,4-tetrahydro-9-isopropyl-1,4-methanonaphthalen-5-yl]pyrazole-4-carboxamide (isopyrazam); piperazine compounds such as triforine; pyridine compounds such as pyrifenox; carbinol compounds such as fenarimol and flutriafol; piperidine compounds such as fenpropidine; morpholine compounds such as fenpropimorph, spiroxamine and tridemorph; organotin compounds such as fentin hydroxide and fentin acetate; urea compounds such as pencycuron; cinnamic acid compounds such as dimethomorph and flumorph; phenylcarbamate compounds such as diethofencarb; cyanopyrrole compounds such as fludioxonil and fenpiclonil; strobilurin compounds such as azoxystrobin, kresoxim-methyl, metominofen, trifloxystrobin, picoxystrobin, oryzastrobin, dimoxystrobin, pyraclostrobin and fluoxastrobin; oxazolidinone compounds such as famoxadone; thiazolecarboxamide compounds such as ethaboxam; silylamide compounds such as silthiopham; aminoacid amidecarbamate compounds such as iprovalicarb, benthiavalicarb-isopropyl and methyl N-(isopropoxycarbonyl)-L-valyl-(3RS)-3-(4-chlorophenyl)-β-alaninate (valifenalate); imidazolidine compounds such as fenamidone; hydroxanilide compounds such as fenhexamid; benzenesulfonamide compounds such as flusulfamide; oxime ether compounds such as cyflufenamid; phenoxyamide compounds such as fenoxanil; antibiotics such as validamycin, kasugamycin and polyoxins; guanidine compounds such as iminoctadine and dodine; quinoline compounds such as 6-tert-butyl-8-fluoro-2,3-dimethylquinolin-4-yl acetate (tebufloquin); thiazolidine compounds such as 2-[2-fluoro-5-(trifluoromethyl)phenylthio]-2-[3-(2-methoxyphenyl)thiazolidin-2-ylidene]acetonitrile (flutianil); and other compounds such as isoprothiolane, pyroquilon, diclomezine, quinoxyfen, propamocarb hydrochloride, chloropicrin, dazomet, metam-sodium, nicobifen, metrafenone, MTF-753, UBF-307, diclocymet, proquinazid, amisulbrom (another name: amibromdole), pyribencarb, mandipropamid, fluopicolide, carpropamid, meptylidinocap, fluopyram, BCF-051, BCM-061 and BCM-062.

Further, agricultural chemicals which may be used in admixture with or in combination with the compounds of the present invention, may, for example, be the active ingredient compounds in the herbicides as disclosed in The Pesticide Manual (14th edition), particularly those of soil treatment type.

The pesticides against parasites on animals are effective for controlling e.g. external parasites which are parasitic on the body surface of host animals (such as the back, the axilla, the lower abdomen or inside of the thigh) or internal parasites which are parasitic in the body of host animals (such as the stomach, the intestinal tract, the lung, the heart, the liver, the blood vessels, the subcutis or lymphatic tissues), but they are particularly effective for controlling the external parasites.

The external parasites may, for example, be animal parasitic acarus or fleas. Their species are so many that it is difficult to list all of them, and therefore, their typical examples will be given.

The animal parasitic acarus may, for example, be ticks such as Boophilus microplus, Rhipicephalus sanguineus, Haemaphysalis longicornis, Haemaphysalis flava, Haemaphysalis campanulata, Haemaphysalis concinna, Haemaphysalis japonica, Haemaphysalis kitaokai, Haemaphysalis ias, Ixodes ovatus, Ixodes nipponensis, Ixodes persulcatus, Amblyomma testudinarium, Haemaphysalis megaspinosa, Dermacentor reticulatus, and Dermacentor taiwanesis; red mite (Dermanyssus gallinae); northern fowl mites such as Ornithonyssus sylviarum, and Ornithonyssus bursa; trombiculidae such as Eutrombicula wichmanni, Leptotrombidium akamushi, Leptotrombidium pallidum, Leptotrombidium fuji, Leptotrombidium tosa, Neotrombicula autumnalis, Eutrombicula alfreddugesi, and Helenicula miyagawai; cheyletidae such as Cheyletiella yasguri, Cheyletiella parasitivorax, and Cheyletiella blakei; sarcoptic mange mites such as Psoroptes cuniculi, Chorioptes bovis, Otodectes cynotis, Sarcoptes scabiei, and Notoedres cati; and Demodicidae such as Demodex canis. The pesticides against parasites on animals, containing the compounds of the present invention, are particularly effective for the control of ticks among them.

The fleas may, for example, be externally parasitic wingless insects belonging to Siphonaptera, more specifically, fleas belonging to Pulicidae, Ceratephyllus, etc. Fleas belonging to Pulicidae may for example, be Ctenocephalides canis, Ctenocephalides felis, Pulex irritans, Echidnophaga gallinacea, Xenopsylla cheopis, Leptopsylla segnis, Nosopsyllus fasciatus, and Monopsyllus anisus. The pesticides against parasites on animals, containing the compounds of the present invention, are particularly effective for the control of fleas belonging to Pulicidae, particularly Ctenocephalides canis and Ctenocephalides felis, among them.

Other external parasites may, for example, be sucking lice (Anoplura) such as shortnosed cattle louse (Haematopinus eurysternus), horse sucking louse (Haematopinus asini), sheep louse, longnosed cattle louse (Linognathus vituli), and head louse (Pediculus capitis); biting lice such as dog biting louse (Trichodectes canis); and blood-sucking dipterous insects such as horsefly (Tabanus trigonus), biting midges (Culicoides schultzei), and blackfly (Simulium ornatum). Further, the internal parasites may, for example, be nematodes such as lung worms, whipworms (Trichuris), tuberous worms, gastric parasites, ascaris, and filarioidea; cestoda such as Spirometra erinacei, Diphyllobothrium latum, Dipylidium caninum, Taenia multiceps, Echinococcus granulosus, and Echinococcus multilocularis; trematoda such as Schistosoma iaponicum and Fasciola hepatica; and protozoa such as coccidia, malaria parasites (Plasmodium malariae), intestinal sarcocyst, toxoplasma, and cryptosporidium.

The host animals may, for example, be pet animals, domestic animals, and poultry, such as dogs, cats, mice, rats, hamsters, guinea pigs, squirrels, rabbits, ferrets, birds (such as pigeons, parrots, hill mynas, Java sparrows, honey parrots, lovebirds and canaries), cows, horses, pigs, sheep, ducks and chickens. The pesticides against parasites on animals, containing the compounds of the present invention, are particularly effective for the control of pests parasitic on pet animals or domestic animals, especially for the control of external parasites, among them. Among pet animals or domestic animals, they are effective particularly for dogs and cats, cows and horses.

When the compound of the present invention is used as a pesticide against parasites on animals, it may be used as it is or may be used together with suitable adjuvants, as formulated into various formulations such as a dust, granules, tablets, a powder, capsules, a soluble concentrate, an emulsifiable concentrate, a water-based suspension concentrate and an oil-based suspension concentrate. In addition to such formulations, it may be formulated into any type of formulation which is commonly used in this field, so long as it is suitable for the purpose of the present invention. The adjuvants to be used for formulations may, for example, be anionic surfactants or nonionic surfactants exemplified above as adjuvants for formulation of agricultural and horticultural pesticides; a cationic surfactant such as cetyl trimethylammonium bromide; a solvent such as water, acetone, acetonitrile, N-methylacetamide, N,N-dimethylacetamide, N,N-dimethylformamide, 2-pyrrolidone, N-methyl-2-pyrrolidone, kerosene, triacetin, methanol, ethanol, isopropanol, benzyl alcohol, ethylene glycol, propylene glycol, polyethylene glycol, liquid polyoxyethylene glycol, butyl diglycol, ethylene glycol monomethyl ether, ethylene glycol monoethyl ether, diethylene glycol monoethyl ether, diethylene glycol n-butyl ether, dipropylene glycol monomethyl ether, or dipropylene glycol n-butyl ether; an antioxidant such as butylhydroxyanisole, butylhydroxytoluene, ascorbic acid, sodium hydrogenmetasulfite, propyl gallate or sodium thiosulfate; a coating film-forming agent such as polyvinylpyrrolidone, polyvinyl alcohol, or a copolymer of vinyl acetate and vinyl pyrrolidone; the vegetable oils and mineral oils as exemplified above as adjuvants for formulation of agricultural and horticultural pesticides; a carrier such as lactose, sucrose, glucose, starch, wheat flour, corn powder, soybean cake and meal, defatted rice bran, calcium carbonate or other commercially available feed materials; and so on. One or more of the respective components of these adjuvants may be suitably selected for use, so long as such will not depart from the purpose of the present invention. Further, other than the above-mentioned adjuvants, some among those known in this field may suitably be selected for use, and still further, some among the above-mentioned various adjuvants to be used in the agricultural and horticultural field may suitably be selected for use.

The blend ratio of the compound of the present invention to various adjuvants is usually from 0.1:99.9 to 90:10, by weight. In the actual use of such a formulation, it may be used as it is, or may be diluted to a predetermined concentration with a diluent such as water, and various spreaders (e.g. surfactants, vegetable oils or mineral oils) may be added thereto, as the case requires.

Administration of the compound of the present invention to a host animal is carried out orally or parenterally. As an oral administration method, a method of administering a tablet, a liquid agent, a capsule, a wafer, a biscuit, a minced meat or other feed, containing the compound of the present invention, may be mentioned. As a parenteral administration method, there may, for example, be mentioned a method wherein the compound of the present invention is formulated into a suitable formulation and then taken into the body by e.g. intravenous administration, intramuscular administration, intradermal administration, hypodermic administration, etc.; a method wherein it is administered on the body surface by spot-on treatment, pour-on treatment or spray treatment; or a method of embedding a resin fragment or the like containing the compound of the present invention under the skin of the host animal. The dose of the compound of the present invention to a host animal varies depending upon the administration method, the purpose of administration, the deceased symptom, etc., but it is usually administered in a proportion of from 0.01 mg to 100 g, preferably from 0.1 mg to 10 g, per 1 kg of the body weight of the host animal.

The present invention also includes a method for controlling a pest by the above-mentioned administration method or by the above-mentioned dose, particularly a method for controlling external parasites or internal parasites.

Further, in the present invention, by controlling pests parasitic on animals as described above, it is possible to prevent or cure various diseases of the host animal thereby caused in some cases. Thus, the present invention also includes a preventive or therapeutic agent for an animal disease caused by parasites, containing the compound of the present invention as an active ingredient, and a method for preventing or curing an animal disease caused by parasites.

When the compound of the present invention is used as a pesticide against parasites on animals, various vitamins, minerals, amino acids, nutrients, enzymes, antipyretics, sedatives, antiphlogistics, fungicides, colorants, aromatic substances, preservatives, etc., may be used in admixture with or in combination with the adjuvants. Further, as the case requires, other animal drugs or agricultural chemicals, such as vermicides, anti-coccidium agents, insecticides, miticides, pulicides, nematicides, bactericides or antibacterial agents, may be mixed or combined for use, whereby improved effects may sometimes be obtained. The present invention includes such a mixed pesticidal composition having the above-mentioned various components mixed or combined for use, and further a method for controlling a pest by using it, particularly a method for controlling external parasites or internal parasites.

Now, preferred embodiments of the present invention will be described, but it should be understood that the present invention is by no means thereby restricted.
(1) A pyridine derivative represented by the formula (I) or its salt, wherein R¹ is alkyl, cycloalkyl, alkoxyalkyl or OR³; R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, or phenoxy which may be substituted by B; R³ is alkyl which may be substituted by D, cycloalkyl which may be substituted by E, alkenyl which may be substituted by D, alkynyl which may be substituted by D, phenylalkyl which may be substituted by E, pyridylalkyl which may be substituted by E, phenyl which may be substituted by E, silyl which is substituted by E, N-alkylcarbamoyl, N-alkoxycarbamoyl or N,N-dialkylcarbamoyl; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; D is at least one substituent selected from the group consisting of cycloalkyl, halogen, alkoxy, haloalkoxy, alkylthio, cyano, nitro, alkylcarbonyl, haloalkylcarbonyl, alkoxycarbonyl and alkylsilyl; E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl, alkylsilyl, tetrahydropyranyl, 1,3-dioxolan-2-yl and N,N-dialkylamino; and m is an integer of from 1 to 4.
(2) The pyridine derivative or its salt according to the above (1), wherein X is alkyl which may be substituted by A, cycloalkyl which may be substituted by B, halogen, nitro, cyano, or alkoxy which may be substituted by A; R³ is alkyl which may be substituted by D, cycloalkyl which may be substituted by E, or alkenyl which may be substituted by D; and E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl and alkylsilyl.
(3) The pyridine derivative or its salt according to the above (2), wherein R¹ is OR³; and R² is 1H-1,2,4-triazol-1-yl, 1H-imidazol-1-yl, 1H-1,2,3-triazol-1-yl or 4H-1,2,4-trizol-4-yl.
(4) A process for producing a pyridine derivative represented by the formula (I) or its salt, wherein R¹ is alkyl, cycloalkyl, alkoxyalkyl or OR³; R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, or phenoxy which may be substituted by B; R³ is alkyl which may be substituted by D, cycloalkyl which may be substituted by E, alkenyl which may be substituted by D, alkynyl which may be substituted by D, phenylalkyl which may be substituted by E, pyridylalkyl which may be substituted by E, phenyl which may be substituted by E, silyl which is substituted by E, N-alkylcarbamoyl, N-alkoxycarbamoyl or N,N-dialkylcarbamoyl; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; D is at least one substituent selected from the group consisting of cycloalkyl, halogen, alkoxy, haloalkoxy, alkylthio, cyano, nitro, alkylcarbonyl, haloalkylcarbonyl, alkoxycarbonyl and alkylsilyl; E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl, alkylsilyl, tetrahydropyranyl, 1,3-dioxolan-2-yl and N,N-dialkylamino; and m is an integer of from 1 to 4; which comprises

(a) reacting a compound represented by the formula (III), wherein Z is halogen; and R¹, X and m are as defined above, with a compound represented by the formula (IV), wherein R² is as defined above; or

(b) reacting a compound represented by the formula (VI), wherein R², X and m are as defined above, with a compound represented by the formula (VII), wherein L is halogen, alkylsulfonyloxy, trifluoromethanesulfonyloxy, or benzenesulfonyloxy which may be substituted by alkyl; and R³ is as defined above.
(5) A compound represented by the formula (VI) or its salt, wherein R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, or phenoxy which may be substituted by B, A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; and m is an integer of from 1 to 4.

### EXAMPLES

Now, the present invention will be described in further detail with reference to Examples, but it should be understood that the present invention is by no means restricted thereto. Firstly, Preparation Examples of the compounds of the present invention will be described.

### Preparation Example 1

### Preparation of N-{[3-chloro-5-(trifluoromethyl)pyridin-2-yl](1H-imidazol-1-yl)methylene}propane-2-amine (Compound No. 1)

(1) To 1.0 g of 3-chloro-5-(trifluoromethyl)picolinic acid, 1.0 ml of thionyl chloride and 0.1 ml of N,N-dimethylformamide were added, followed by heating and refluxing for 3 hours. After completion of the reaction, the reaction mixture was concentrated under reduced pressure. A mixture of the obtained residue and 1 ml of tetrahydrofuran was dropwise added to a mixture of 0.52 g of isopropylamine and 10 ml of tetrahydrofuran under cooling with ice, followed by stirring for 1 hour under cooling with ice. After completion of the reaction, the reaction mixture was extracted with ethyl acetate and washed with a saturated aqueous sodium chloride solution. The organic layer was dried over anhydrous sodium sulfate and then concentrated under reduced pressure. The residue was subjected to washing with hexane to obtain 1.05 g of 3-chloro-N-isopropyl-5-(trifluoromethyl)picolinamide as colorless needle crystals. Its NMR spectrum data are as follows.

¹H NMR (400 MHz, CDCl₃): δ ppm=1.27(6H, d, J=6.4 Hz), 4.19-4.28(1H, m), 7.49(1H, broad singlet), 8.03(1 H, d, J=1.2 Hz), 8.67(1 H, d, J=1.2 Hz)
(2) To a mixture of 0.50 g of 3-chloro-N-isopropyl-5-(trifluoromethyl)picolinamide and 5 ml of toluene, 0.39 g of phosphorus pentachloride was added, followed by heating and refluxing for 3 hours. After completion of the reaction, the reaction mixture was concentrated under reduced pressure to obtain 1.1 ml of an oil containing 3-chloro-N-isopropyl-5-(trifluoromethyl)picolinimidoyl chloride.
(3) To a mixture of 0.10 g of imidazole and 2 ml of acetonitrile, 0.4 ml of the oil obtained in (2) was dropwise added at room temperature, followed by stirring for 1.5 hours at room temperature.

After completion of the reaction, water was added to the reaction mixture, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 0.11 g of the desired product as yellow crystals.

### Preparation Example 2

### Preparation of [3-chloro-5-(trifluoromethyl)pyridin-2-yl](1H-1,2,4-triazol-1-yl)methanone O-oxime (Compound No. 10)

(1) To 2.0 g of 3-chloro-5-(trifluoromethyl)picolinic acid, 2.0 ml of thionyl chloride and 0.2 ml of N,N-dimethylformamide were added, followed by heating and refluxing for 2 hours. After completion of the reaction, the reaction mixture was concentrated under reduced pressure. A mixture of the obtained residue and 1 ml of tetrahydrofuran was dropwise added to a mixture of 0.95 g of O-ethylhydroxylamine hydrochloride, 1.99 g of triethylamine, 10 ml of tetrahydrofuran and 10 ml of N,N-dimethylformamide, under cooling with ice, followed by stirring for 1 hour at room temperature. After completion of the reaction, the reaction mixture was put into water, extracted with ethyl acetate and washed with a saturated aqueous sodium chloride solution. The organic layer was dried over anhydrous sodium sulfate and then concentrated under reduced pressure. The residue was subjected to washing with hexane to obtain 2.20 g of 3-chloro-N-ethoxy-5-(trifluoromethyl)picolinamide as colorless needle crystals. Its NMR spectrum data are as follows.

¹H NMR (400 MHz, CDCl₃): δ ppm=1.33(3H, t, J=7.0 Hz), 4.10(2H, q, J=6.9 Hz), 8.05(1H, s), 8.66(1H, s), 9.82(1H, s)
(2) To a mixture of 0.50 g of 3-chloro-N-ethoxy-5-(trifluoromethyl)picolinamide and 10 ml of acetonitrile, 0.98 g of triphenylphosphine and 0.3 ml of carbon tetrachloride were added, followed by heating and refluxing for 15 hours. After completion of the reaction, the reaction mixture was concentrated under reduced pressure, and the residue was purified by silica gel column chromatography (eluent: n-hexane/ethyl acetate=7/1) to obtain 0.13 g of 3-chloro-N-ethoxy-5-(trifluoromethyl)picolinimidoyl chloride. Its NMR spectrum data are as follows.

¹H NMR (400 MHz, CDCl₃): δ ppm=1.37(3H, t, J=7.6 Hz), 4.37(2H, q, J=7.1 Hz), 8.03(1H, s), 8.81(1H, s)
(3) To a mixture of 32 mg of 1,2,4-triazole and 5 ml of N,N-dimethylformamide, 19 mg of sodium hydride (60 wt % dispersion in mineral oil) was added under cooling with ice, followed by stirring at room temperature for 15 minutes. Then, 90 mg of 3-chloro-N-ethoxy-5-(trifluoromethyl)picolinimidoyl chloride was dropwise added at room temperature, followed by stirring at 100° C. for 20 hours. After completion of the reaction, the reaction mixture was returned to room temperature, and water was added, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 5 mg of the desired product as a colorless oil.

### Preparation Example 3

### Preparation of [3-chloro-5-(trifluoromethyl)pyridin-2-yl](1H-1,2,4-triazol-1-yl)methanone O-isopropyl oxime (Compound No. 13)

(1) To a mixture of 3.0 g of 3-chloro-5-(trifluoromethyl)picolinaldehyde, 30 ml of methanol and 30 ml of water, a mixture of 1.2 g of hydroxylamine hydrochloride, 0.91 g of sodium carbonate and 10 ml of water, was dropwise added at room temperature, followed by stirring at room temperature for 30 minutes. After completion of the reaction, 30 ml of water was added to the reaction mixture, followed by stirring at room temperature for 30 minutes. Precipitated crystals were collected by filtration, washed with water and dried to obtain 2.19 g of 3-chloro-5-(trifluoromethyl)picolinaldehyde oxime as colorless crystals. Its NMR spectrum data are as follows.

¹H NMR (400 MHz, CDCl₃): δ ppm=7.68(1H, s), 8.36(1H, s), 8.52(1H, s), 9.15(1H, s)
(2) To a mixture of 1.0 g of 3-chloro-5-(trifluoromethyl)picolinaldehyde oxime and 5 ml of N,N-dimethylformamide, 0.67 g of N-chlorosuccinimide was added, and hydrochloric acid gas was blown thereinto for 2 seconds, followed by stirring at room temperature for 1 hour. After completion of the reaction, water was added to the reaction mixture, followed by extraction with diethyl ether and washing with a saturated aqueous sodium chloride solution. The organic layer was dried over anhydrous sodium sulfate and then concentrated under reduced pressure to obtain 1.20 g of 3-chloro-N-hydroxy-5-(trifluoromethyl)picolinimidoyl chloride as an oil. Its NMR spectrum data are as follows.

¹H NMR (400 MHz, CDCl₃): δ ppm=8.05(1H, s), 8.79(1H, s), 9.58(1H, s)
(3) To a mixture of 0.40 g of 1,2,4-triazole and 10 ml of N,N-dimethylformamide, 0.23 g of sodium hydride (60 wt % dispersion in mineral oil) was added under cooling with ice, followed by stirring at room temperature for 30 minutes. Then, a mixture of 1.0 g of 3-chloro-N-hydroxy-5-(trifluoromethyl)picolinimidoyl chloride and 5 ml of N,N-dimethylformamide, was dropwise added under cooling with ice, followed by stirring at room temperature for 2 hours. After completion of the reaction, water was added to the reaction mixture, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 0.29 g of [3-chloro-5-(trifluoromethyl)pyridin-2-yl](1H-1,2,4-triazol-1-yl)methanone oxime (Compound No. VI-2) as a colorless amorphous solid.
(4) To a mixture of 0.20 g of [3-chloro-5-(trifluoromethyl)pyridin-2-yl](1H-1,2,4-triazol-1-yl)methanone oxime and 4 ml of N,N-dimethylformamide, 30 mg of sodium hydride (60 wt % dispersion in mineral oil) was added under cooling with ice, followed by stirring at room temperature for 15 minutes. Then, a mixture of 0.17 g of isopropyl iodide and 1 ml of N,N-dimethylformamide, was dropwise added under cooling with ice, followed by further stirring at room temperature for 1.5 hours. After completion of the reaction, water was added to the reaction mixture, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 0.19 g of the desired product as a colorless oil.

### Preparation Example 4

### Preparation of [3-methyl-5-(trifluoromethyl)pyridin-2-yl](1H-1,2,4-triazol-1-yl)methanone O-ethyl oxime (Compound No.33)

(1) To a mixture of 0.53 g of 3-methyl-5-(trifluoromethyl)picolinonitrile and 10 ml of ethanol, a mixture of 0.22 g of hydroxylamine hydrochloride, 0.17 g of sodium carbonate and 10 ml of water, was added, followed by heating and refluxing for 1 hour. After completion of the reaction, the reaction mixture was concentrated under reduced pressure, and 50 ml of water was added, followed by stirring at room temperature. Precipitated crystals were collected by filtration, washed with water and dried to obtain 0.58 g of N′-hydroxy-3-methyl-5-(trifluoromethyl)picolinimidamide as colorless crystals. Its NMR spectrum data are as follows.

¹H NMR (400 MHz, CDCl₃): δ ppm=2.62(3H, s), 5.63(2H, broad singlet), 7.75(1H,s), 8.69(1H, s)
(2) To a mixture of 0.58 g of N′-hydroxy-3-methyl-5-(trifluoromethyl)picolinimidamide and 10 ml of a 10 wt % hydrochloric acid aqueous solution, a mixture of 0.22 g of sodium nitrite and 2 ml of water, was dropwise added under cooling with ice, followed by stirring for 1 hour under cooling with ice. After completion of the reaction, the reaction mixture was extracted with ethyl acetate and washed with a saturated aqueous sodium chloride solution. The organic layer was dried over anhydrous sodium sulfate and then concentrated under reduced pressure to obtain 0.57 g of N-hydroxy-3-methyl-5-(trifluoromethyl)picolinimidoyl chloride as a solid. Its NMR spectrum data are as follows.

¹H NMR (400 MHz, CDCl₃): δ ppm=2.47(3H, s), 7.79(1H, s), 8.72(1H, s)
(3) To a mixture of 0.20 g of 1,2,4-triazole and 20 ml of N,N-dimethylformamide, 116 mg of sodium hydride (60 wt % dispersion in mineral oil) was added under cooling with ice, followed by stirring at room temperature for 30 minutes. Then, a mixture of 0.57 g of N-hydroxy-3-methyl-5-(trifluoromethyl)picolinimidoyl chloride and 10 ml of N,N-dimethylformamide was dropwise added under cooling with ice, followed by stirring for 30 minutes under cooling with ice and further stirring at room temperature for 30 minutes. After completion of the reaction, the reaction mixture was put into water, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 0.13 g of [3-methyl-5-(trifluoromethy)pyridin-2-yl](1H-1,2,4-triazol-1-yl)methanone oxime (Compound No. VI-5) as a colorless amorphous solid.
(4) To a mixture of 0.13 g of [3-methyl-5-(trifluoromethyl)pyridin-2-yl](1H-1,2,4-triazol-1-yl)methanone oxime and 4 ml of N,N-dimethylformamide, 21 mg of sodium hydride (60 wt % dispersion in mineral oil) was added under cooling with ice, followed by stirring at room temperature for 30 minutes. Then, a mixture of 0.11 g of ethyl iodide and 1 ml of N,N-dimethylformamide was dropwise added under cooling with ice, followed by stirring at room temperature for 1.5 hours. After completion of the reaction, water was added to the reaction mixture, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 72 mg of the desired product as a colorless oil.

### Preparation Example 5

### Preparation of [3-methylthio-5-(trifluoromethyl)pyridin-2-yl](1H-1,2,4-triazol-1-yl)methanone O-ethyl oxime (Compound No. 67)

To a mixture of 0.10 g of [3-chloro-5-(trifluoromethyl)pyridin-2-yl]((1H-1,2,4-triazol-1-yl)methanone O-ethyl oxime (Compound No. 10) and 2 ml of dimethylsulfoxide, 25 mg of sodium thiomethoxide was added at room temperature, followed by stirring at 80° C. for 15 hours. After completion of the reaction, water was added to the reaction mixture, followed by extraction with ethyl acetate and washing with a saturated aqueous sodium chloride solution. The organic layer was dried over anhydrous sodium sulfate and then concentrated under reduced pressure. The residue was purified by silica gel flash chromatography (eluent: n-hexane/ethyl acetate) to obtain 74 mg of the desired product as a colorless oil.

Typical examples of the compound of the above formula (I) will be given in Table 1. These compounds can be prepared by the above-described Preparation Examples or by the above-described various processes for the production of the compound of the present invention. In Table 1, No. represents the Compound No., Me methyl, Et ethyl, n-Pr normal propyl, i-Pr isopropyl, n-Bu normal butyl, t-Bu tertiary butyl, sec-Bu secondary butyl and Ph phenyl, and the temperature shown as the physical properties is the melting point. Further, with respect to some compounds of the above formula (I), ¹H-NMR is shown in Table 2.

The compound of the above formula (VI) includes novel compounds, and typical examples thereof will be given in Table 3. These compounds can be prepared by the above-described Preparation Examples or by the above-described production processes. Further, the compound of the formula (VI) can form a salt, and such a salt includes all kinds so long as they are acceptable in this technical field, and it may, for example, be an alkali metal salt such as a sodium salt or a potassium salt; an alkaline earth metal salt such as a magnesium salt or a calcium salt; an inorganic acid salt such as a hydrochloride, a perchlorate, a sulfate or a nitrate; or an organic acid salt such as an acetate or a methanesulfonate. In Table 3, No. represents the Compound No., Me methyl and t-Bu tertiary butyl, and the temperature shown as the physical properties is the melting point. Further, with respect to some compounds of the above formula (VI), ¹H-NMR is shown in Table 4.

TABLE 1

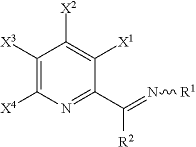

| | | | | | | | Physical |
|---|---|---|---|---|---|---|---|
| No. | R¹ | R² | X¹ | X² | X³ | X⁴ | properties |
| 1 | i-Pr | 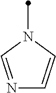 | Cl | H | CF₃ | H | 52-54° C. |
| 2 | i-Pr | 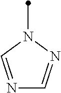 | Cl | H | CF₃ | H | oil |
| 3 | Et | 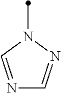 | Cl | H | CF₃ | H | |
| 4 | Me | 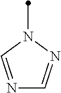 | Cl | H | CF₃ | H | |
| 5 | n-Pr | 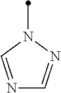 | Cl | H | CF₃ | H | |
| 6 | 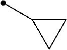 | 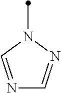 | Cl | H | CF₃ | H | |
| 7 | CH₂OMe | 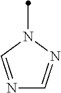 | Cl | H | CF₃ | H | |
| 8 | CH₂CH₂OMe | 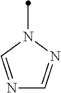 | Cl | H | CF₃ | H | amorphous |
| 9 | CH₂OEt | 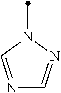 | Cl | H | CF₃ | H | |
| 10 | OEt | 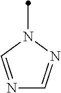 | Cl | H | CF₃ | H | oil |
| 11 | OEt | 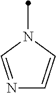 | Cl | H | CF₃ | H | oil |
| 12 | OEt | 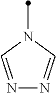 | Cl | H | CF₃ | H | 75-76° C. |
| 13 | O(i-Pr) | 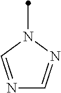 | Cl | H | CF₃ | H | oil |
| 14 | OMe | 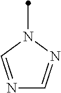 | Cl | H | CF₃ | H | 83-84° C. |
| 15 | O(sec-Bu) | 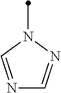 | Cl | H | CF₃ | H | oil |
| 16 | O(n-Pr) | 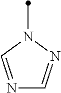 | Cl | H | CF₃ | H | |
| 17 | O(n-Bu) | 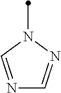 | Cl | H | CF₃ | H | |
| 18 | O(t-Bu) | 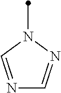 | Cl | H | CF₃ | H | |
| 19 | 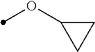 | 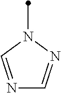 | Cl | H | CF₃ | H | |
| 20 | OEt | 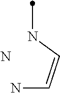 | Cl | H | CF₃ | H | oil |
| 21 | OCH₂Ph | 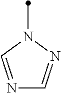 | Cl | H | CF₃ | H | 89-90° C. |
| 22 | OCH₂CH₂OMe | 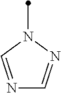 | Cl | H | CF₃ | H | oil |
| 23 | OEt | 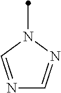 | H | H | H | Me | 69-70° C. |
| 24 | OEt | 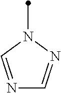 | H | H | CF₃ | H | oil |
| 25 | OEt | 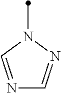 | CF₃ | H | H | H | 89-90° C. |
| 26 | OEt | 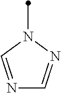 | H | CF₃ | H | H | 70-71° C. |
| 27 | OEt | 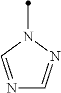 | H | H | H | CH₂CH₂(t-Bu) | oil |
| 28 | OCH₂CN | 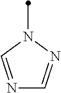 | Cl | H | CF₃ | H | oil |
| 29 | OCH₂COOMe | 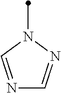 | Cl | H | CF₃ | H | oil |
| 30 | OCH₂CH═CH₂ | 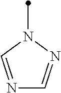 | Cl | H | CF₃ | H | oil |
| 31 | OEt | 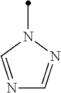 | Br | H | CF₃ | H | oil |
| 32 | 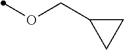 | 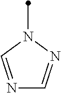 | Cl | H | CF₃ | H | oil |
| 33 | OEt | 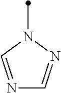 | Me | H | CF₃ | H | oil |
| 34 | OEt | 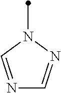 | H | Me | CF₃ | H | |
| 35 | OEt | 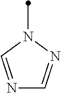 | H | CH₂CH₂(t-Bu) | CF₃ | H | |
| 36 | OEt | 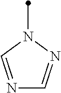 | Cl | H | H | CH₂CH₂(t-Bu) | |
| 37 | OEt | 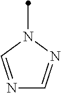 | Cl | CH₂CH₂(t-Bu) | H | H | |
| 38 | OEt | 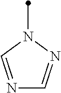 | 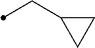 | H | CF₃ | H | |
| 39 | O(i-Pr) | 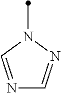 | 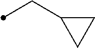 | H | CF₃ | H | |
| 40 | OEt | 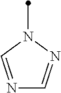 | CH₂OMe | H | CF₃ | H | |
| 41 | OEt | 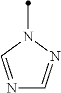 | H | H | H | CH₂OMe | |
| 42 | OEt | 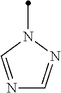 | CH₂OEt | H | CF₃ | H | |
| 43 | OEt | 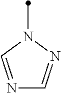 | CH₂O(n-Pr) | H | CF₃ | H | |
| 44 | OEt | 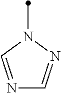 | CH₂O(i-Pr) | H | CF₃ | H | |
| 45 | OEt | 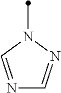 | CH₂CH₂OEt | H | CF₃ | H | |
| 46 | OEt | 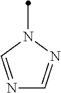 | CH₂CH₂OMe | H | CF₃ | H | |
| 47 | OEt | 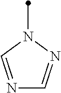 | H | H | H | 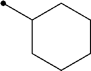 | |
| 48 | OEt | 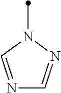 | H | H | H | 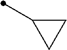 | |
| 49 | OEt | 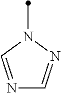 | H | H | H | 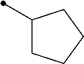 | |
| 50 | OEt | 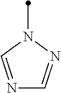 | NO₂ | H | CF₃ | H | |
| 51 | OEt | 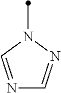 | CN | H | CF₃ | H | |
| 52 | OEt | 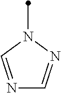 | OMe | H | CF₃ | H | |
| 53 | O(i-Pr) | 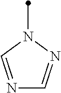 | OMe | H | CF₃ | H | |
| 54 | OEt | 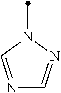 | OEt | H | CF₃ | H | |
| 55 | OEt | 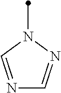 | O(n-Pr) | H | CF₃ | H | |
| 56 | OEt | 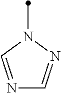 | O(n-Bu) | H | CF₃ | H | |
| 57 | OEt | 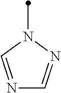 | | H | CF₃ | H | |
| 58 | OEt | 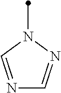 | | H | CF₃ | H | |
| 59 | OEt | 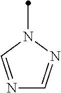 | OCH₂CH₂OMe | H | CF₃ | H | |
| 60 | O(i-Pr) | 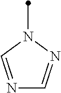 | OCH₂CH₂OMe | H | CF₃ | H | |
| 61 | OEt | 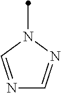 | H | H | H | OCH₂CH₂OMe | |
| 62 | O(i-Pr) | 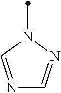 | H | H | H | OCH₂CH₂OMe | |
| 63 | OEt | 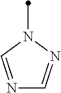 | OCH₂CH₂OCF₃ | H | CF₃ | H | |
| 64 | OEt | 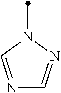 | OCH₂Ph | H | CF₃ | H | |
| 65 | OEt | 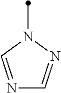 | CH₂SiMe₃ | H | CF₃ | H | |
| 66 | OEt | 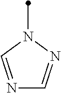 | OCH₂SiMe₃ | H | CF₃ | H | |
| 67 | OEt | 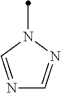 | SMe | H | CF₃ | H | oil |
| 68 | OEt | 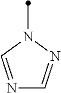 | H | H | H | CH═CH(t-Bu) | |
| 69 | OEt | 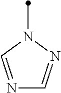 | H | H | H | C≡CH | |
| 70 | OEt | 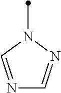 | OPh | H | CF₃ | H | |
| 71 | OEt | 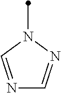 | Cl | H | CF₃ | H | |
| 72 | OEt | 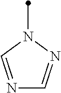 | Cl | H | CF₃ | H | |
| 73 | OCH₂CF₃ | 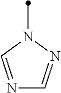 | Cl | H | CF₃ | H | |
| 74 | OCH₂SMe | 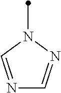 | Cl | H | CF₃ | H | |
| 75 | OCH₂COMe | 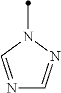 | Cl | H | CF₃ | H | |
| 76 | OCH₂COCF₃ | 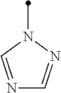 | Cl | H | CF₃ | H | |
| 77 | OCH₂SiMe₃ | 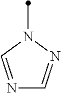 | Cl | H | CF₃ | H | |
| 78 | 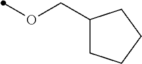 | 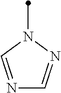 | Cl | H | CF₃ | H | |
| 79 | 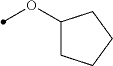 | 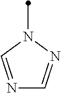 | Cl | H | CF₃ | H | |
| 80 | OCH₂CH═CHCH₃ | 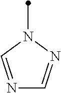 | Cl | H | CF₃ | H | |
| 81 | OCH₂CHC═Cl₂ | 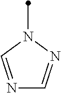 | Cl | H | CF₃ | H | |
| 82 | OCH₂CH═CHCl | 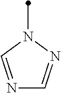 | Cl | H | CF₃ | H | |
| 83 | OEt | 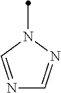 | H | H | H | CF₃ | 67-68° C. |
| 84 | OEt | 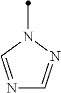 | Cl | H | H | CF₃ | 93-94° C. |
| 85 | CH₂CH₂OMe | 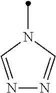 | Cl | H | CF₃ | H | oil |
| 86 | OEt | 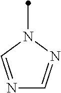 | Cl | H | H | OCH₂(t-Bu) | |
| 87 | O(i-Pr) | 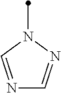 | Cl | H | H | CH₂CH₂(t-Bu) | |
| 88 | OEt | 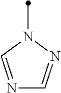 | Cl | H | H | CH═CH(t-Bu) | |
| 89 | OEt | 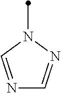 | Cl | H | H | C≡CH(t-Bu) | |
| 90 | OEt | 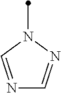 | F | H | CF₃ | H | |
| 91 | OEt | 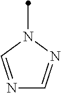 | NH₂ | H | CF₃ | H | |
| 92 | OEt | 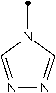 | NHCOCF₃ | H | CF₃ | H | |
| 93 | OEt | 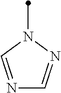 | 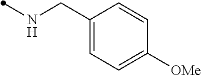 | H | CF₃ | H | |
| 94 | OEt | 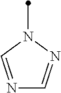 | Cl | H | H | Cl | |
| 95 | OEt | 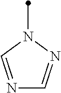 | Cl | H | H | OCH₂SiMe₃ | |
| 96 | OEt | 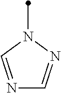 | Cl | H | H | OCOCF₃ | |
| 97 | OEt | 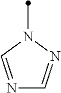 | NMe2 | H | CF₃ | H | |
| 98 | OEt | 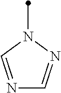 | NHCH₂OMe | H | CF₃ | H | |
| 99 | OEt | 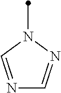 | NHCH₂CF₃ | H | CF₃ | H | |
| 100 | OEt | 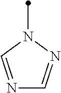 | NHCH₂CN | H | CF₃ | H | |
| 101 | OEt | 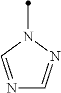 | NHCOOMe | H | CF₃ | H | |
| 102 | OEt | 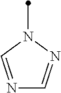 | NHSOMe | H | CF₃ | H | |
| 103 | OEt | 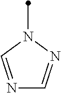 | NHSO₂Me | H | CF₃ | H | |
| 104 | OEt | 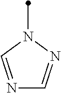 | Cl | H | H | NHCOPh | |
| 105 | OEt | 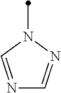 | Cl | H | H | 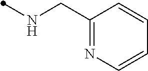 | |
| 106 | OEt | 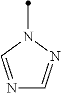 | Cl | H | H | OCOPh | |
| 107 | OEt | 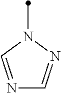 | Cl | H | H | OCOOMe | |
| 108 | OEt | 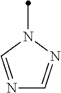 | Cl | H | H | OSOMe | |
| 109 | OEt | 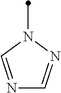 | Cl | H | H | OSO₂Me | |
| 110 | OEt | 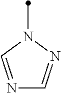 | Cl | H | H | 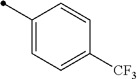 | |
| 111 | OEt | 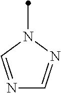 | Cl | H | H | Ph | |
| 112 | OEt | 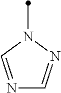 | Cl | H | H | 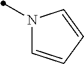 | |
| 113 | OEt | 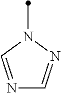 | Cl | H | H | COMe | |
| 114 | OEt | 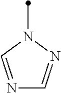 | Cl | H | H | COCF₃ | |
| 115 | OEt | 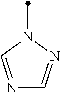 | Cl | H | H | COPh | |
| 116 | OEt | 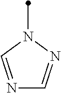 | Cl | H | H | COOPh | |
| 117 | OEt | 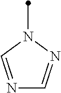 | Cl | H | H | SMe | |
| 118 | OEt | 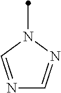 | Cl | H | H | SOMe | |
| 119 | OEt | 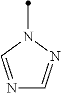 | Cl | H | H | SO₂Me | |
| 120 | OEt | 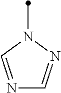 | Cl | H | H | CONMe₂ | |
| 121 | OEt | 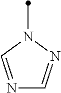 | Cl | H | H | CONHCH₂CN | |
| 122 | OEt | 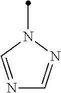 | Cl | H | H | SCH₂CF₃ | |
| 123 | OEt | 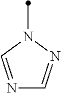 | Cl | H | H | OH | |
| 124 | OEt | 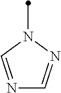 | I | H | CF₃ | H | |

TABLE 2

| N° | ¹H-NMR δ ppm (Solvent: CDCl₃/400 MHz) |
|---|---|
| 2 | 1.20(6H, d, J = 9.6 Hz), 3.35(1H, m), 7.90(1H, s), 8.08(1H, d, J = 2.0 Hz), 8.88(1H, d, J = 1.2 Hz), |
| | 9.05(1H, s) |
| 8 | 3.39(3H, s), 3.53(2H, t, J = 6.0 Hz), 3.69(2H, t, J = 6.0 Hz), 7.93(1H, s), 8.11(1H, d, J = 1.6 Hz), |
| | 8.91(1H, d, J = 1.6 Hz), 9.09(1H, s) |
| 10 | 1.43(3H, t, J = 7.0 Hz), 4.43(2H, q, J = 7.2 Hz), 7.92(1H, s), 8.05(1H, d, J = 1.6 Hz), 8.83(1H, d, |
| | J = 1.2 Hz), 9.30(1H, s) |
| 11 | 1.34(3H, t, J = 7.2 Hz), 4.31(2H, q, J = 6.8 Hz), 7.02(2H, m), 8.03(2H, m), 8.80(1H, s) |
| 13 | 1.41(6H, d, J = 6.4 Hz), 4.63(1H, m), 7.92(1H, s), 8.06(1H, s), 8.83(1H, s), 9.32(1H, s) |
| 15 | 0.98(3H, t, J = 7.2 Hz), 1.38(3H, d, J = 6.4 Hz), 4.44(1H, m), 7.92(1H, s), 8.05(1H, s), 8.83(1H, s), |
| | 9.31(1H, s) |
| 20 | 1.41(3H, t, J = 7.2 Hz), 4.43(2H, q, J = 6.9 Hz), 7.80 (1H, s), 8.04(1H, d, J = 2.0 Hz), 8.71(1H, s), |
| | 8.83(1H, d, J = 1.6 Hz) |
| 22 | 3.41(3H, s), 3.37(2H, m), 4.50(2H, m), 7.92(1H, s), 8.04(1H, broad singlet), 8.83(1H, broad |
| | singlet), 9.35(1H, s) |
| 24 | 1.37(3H, t, J = 7.2 Hz), 4.42(2H, q, J = 7.1 Hz), 8.09(1H, d, J = 8.4 Hz), 8.16(1H, s), 8.25(1H, dd, |
| | J = 8.6 Hz, J = 2.2 Hz), 8.85(1H, broad singlet), 9.01(1H, s) |
| 27 | 0.91(9H, s), 1.36(3H, t, J = 7.2 Hz), 1.53(2H, m), 2.73(2H, m), 4.36(2H, q, J = 7.1 Hz), 7.23(1H, d, |
| | J = 8.0 Hz), 7.52(1H, d, J = 7.2 Hz), 7.67(1H, t, J = 7.8 Hz), 8.02(1H, s), 8.90(1H, s) |
| 28 | 5.00(2H, s), 7.96(1H, s), 8.08(1H, d, J = 0.8 Hz), 8.84(1H, d, J = 1.6 Hz), 9.17(1H, s) |
| 29 | 3.79(3H, s), 4.90(2H, s), 7.93(1H, s), 8.04(1H, s), 8.82(1H, s), 9.41(1H, s) |
| 30 | 4.85(2H, dd, J = 6.6 Hz, J = 1.2 Hz), 5.40(2H, dt, J = 27.6 Hz, J = 10.4 Hz), 6.07(1H, m), 7.92(1H, s), |
| | 8.05(1H, d, J = 1.6 Hz), 8.83(1H, d, J = 0.8 Hz), 9.30(1H, s) |
| 31 | 1.42(3H, t, J = 6.8 Hz), 4.43(2H, q, J = 6.8 Hz), 7.94(1H, s), 8.24(1H, s), 8.87(1H, s), 9.31(1H, s) |
| 32 | 0.38(2H, m), 0.67(2H, m), 1.29(1H, m), 4.20(2H, d, J = 7.2 Hz), 7.95(1H, s), 8.06(1H, d, |
| | J = 2.0 Hz), 8.85(1H, d, J = 1.2 Hz), 9.40(1H, s) |
| 33 | 1.40(3H, t, J = 7.2 Hz), 2.45(3H, s), 4.39(2H, q, J = 7.2 Hz), 7.86(1H, s), 7.92(1H, s), 8.74(1H, s), |
| | 9.27(1H, s) |
| 67 | 1.45(3H, t, J = 7.0 Hz), 2.50(3H, s), 4.44(2H, q, J = 7.0 Hz), 7.84(1H, s), 7.97(1H, s), 8.66(1H, s), |
| | 9.24(1H, s) |
| 85 | 3.34(3H, s), 3.49(2H, t, J = 5.6 Hz), 3.66(2H, t, J = 5.6 Hz), 8.21(1H, s), 8.41(2H, s), 8.99(1H, s) |

TABLE 3

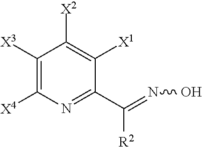

| | | | | | | Physical |
|---|---|---|---|---|---|---|
| No. | R² | X¹ | X² | X³ | X⁴ | properties |
| VI-1 | 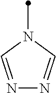 | Cl | H | CF₃ | H | amorphous |
| VI-2 | 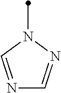 | Cl | H | CF₃ | H | amorphous |
| VI-3 | 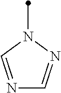 | H | H | H | Me | powder |
| VI-4 | 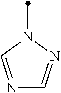 | Br | H | CF₃ | H | powder |
| VI-5 | 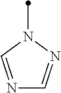 | Me | H | CF₃ | H | amorphous |
| VI-6 | 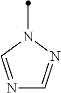 | CF₃ | H | H | H | |
| VI-7 | 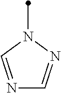 | H | CF₃ | H | H | |
| VI-8 | 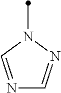 | H | H | CF₃ | H | |
| VI-9 | 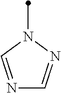 | H | H | H | CH₂CH₂(t-Bu) | |
| VI-10 | 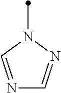 | Cl | H | H | CF₃ | amorphous |
| VI-11 | 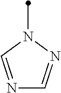 | Cl | H | H | C≡C(t-Bu) | oil |
| VI-12 | 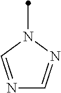 | H | H | H | CF₃ | powder |
| VI-13 | 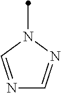 | Cl | H | H | OH | |
| VI-14 | 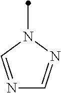 | Cl | H | H | Cl | |

TABLE 4

| No. | ¹H-NMR δ ppm (Solvent: CD₃OD/400 MHz) |
|---|---|
| VI-1 | 8.45(1H, s), 8.89(1H, s), 8.93(2H, s) |
| VI-2 | 8.02(1H, s), 8.41(1H, s), 8.89(1H, s), 9.54(1H, s) |
| VI-3 | 2.47(3H, s), 7.34(1H, d, J = 8.0 Hz), 7.55(1H, d, J = 7.2 Hz), 7.77(1H, t, J = 7.8 Hz), 8.12(1H, s), |
| | 9.05(1H, s) |
| VI-4 | 8.02(1H, s), 8.54(1H, s), 8.93(1H, s), 9.55(1H, s) |
| VI-5 | 2.24(3H, s), 7.83(1H, s), 7.90(1H, s), 8.68(1H, s), 9.40(1H, s), 12.5(1H, broad singlet) |
| | (Solvent: CDCl₃) |
| VI-10 | 7.77(1H, d, J = 8.8 Hz), 8.00(1H, d, J = 8.0 Hz), 8.00(1H, s), 9.54(1H, s), 11.83(1H, broad |
| | singlet) (Solvent: CDCl₃) |
| VI-11 | 1.35(9H, s), 7.45(1H, d, J = 8.8 Hz), 7.73(1H, d, J = 8.4 Hz), 7.96(1H, s), 9.38(1H, s), |
| | 10.74(1H, broad singlet) (Solvent: CDCl₃) |

Now, Test Examples will be described.

### Test Example 1

Test on Controlling Effects Against Green Peach Aphid (Myzus persicae)

A Japanese radish leaf was inserted in a test tube in which water was put, and about 20 first instar nymphs of green peach aphid were released on the leaf. On the next day, the number of nymphs parasitic on the leaf was counted, and then the leaf was dipped for about 10 seconds in an insecticidal solution adjusted to bring the concentration of the compound of the present invention to 200 ppm, dried in air and left in a constant temperature chamber at 25° C. with lightening. Dead nymphs were counted 5 days after the treatment, and the mortality was calculated by the following equation. The insects dropped from the leaf or presented toxic symptom were counted as dead insects. The test was carried out with respect to the above-mentioned compound Nos. 10, 11, 14, 27, 31, 32 and 33, whereby all compounds showed a mortality of at least 90%.

Mortality (%)=(1−(number of survived insects/number of treated insects))×100

### Test Example 2

Test on Controlling Effects Against Brown Planthopper (Nilaparvata lugens)

Rice seedling was dipped for about 10 seconds in an insecticidal solution adjusted to bring the concentration of the compound of the present invention to 200 ppm and then dried in air, its root was wrapped with a wet absorbent cotton, and the seedling was put into a test tube. Then, 10 second-third instar nymphs of Brown Planthopper were released therein, and the test tube was covered with a gauze and left in a constant temperature chamber at 25° C. with lightening. On the 5th day after the release, dead nymphs were counted, and the mortality was calculated by the following equation.

The test was carried out with respect to the above-mentioned Compound Nos. 10, 11, 12, 13, 14, 15, 22, 23, 24, 25, 26, 27, 28, 30, 31, 32, 33, 83 and 84, whereby all compounds showed a mortality of at least 90%.

Mortality (%)=(number of dead insects/number of released insects)×100

### Test Example 3

Test on Controlling Effects Against Silverleaf Whitefly (Bemisia amentifolii)

An insecticidal solution adjusted to bring the concentration of the compound of the present invention to 200 ppm was applied by a hand spray to cucumber seedling planted in a pot on which first-second instar nymphs of silverleaf whitefly were parasitic, and dried in air. Thereafter, the cucumber seedling was left in a constant temperature chamber at 25° C. with lightening. The number of old instar nymphs was counted 7 days after the treatment, and the protective value (%) was obtained by the following equation. The test was carried out with respect to the above-mentioned compound Nos. 10, 31, 32 and 84, whereby all the compounds showed a protective value of at least 80%.

Protective value (%)=(1−((Ta×Cb)/(Tb×Ca)))×100

Ta: The number of old instar nymphs after the treatment at the treated cucumber seedling

Tb: The number of first-second instar nymphs before the treatment at the treated cucumber seedling

Ca: The number of old instar nymphs after the treatment at the untreated cucumber seedling

Cb: The number of first-second instar nymphs before the treatment at the untreated cucumber seedling

### Test Example 4

Pesticidal Test Against Haemaphysalis longicornis Employing a Dog

A gelatin capsule containing the compound of the present invention at a dose of 10 mg/kg weight is applied to a dog (Beagle, 8 months old), and immediately after the application, about 50 young mites of Haemaphysalis longicornis are released on the auricle of the dog and artificially parasitized. After the treatment, observation is carried out to inspect the parasitic number, the fallen number and the mortality of the fallen Haemaphysalis longicornis. As a result, the compound of the present invention is effective to have the parasitized Haemaphysalis longicornis fallen or dead.

### Test Example 5

Pesticidal Test Against Cat Flea (Ctenocephalides felis) Employing a Dog

A gelatin capsule containing the compound of the present invention at a dose of 10 mg/kg weight is applied to a dog (Beagle, 8 months old), and immediately after the application, about 100 non-bloodsucked adults of cat flea are released on the dorsal fur of the dog and artificially parasitized. After the treatment, the cat flea is recovered by means of a flea catching comb, and the parasitized number is counted. As a result, the compound of the present invention is effective to control the parasitizing of cat flea.

Now, Formulation Examples are described below.

### Formulation Example 1

| | |
|---|---|
| (1) Compound of the present invention | 20 parts by weight |
| (2) Clay | 70 parts by weight |
| (3) White carbon | 5 parts by weight |
| (4) Sodium polycarboxylate | 3 parts by weight |
| (5) Sodium alkylnaphthalene sulfonate | 2 parts by weight |

The above components are uniformly mixed to obtain a wettable powder.

### Formulation Example 2

| | | |
|---|---|---|
| (1) Compound of the present invention | 5 | parts by weight |
| (2) Talc | 60 | parts by weight |
| (3) Calcium carbonate | 34.5 | parts by weight |
| (4) Liquid paraffin | 0.5 | part by weight |

The above components are uniformly mixed to obtain a dust.

### Formulation Example 3

| | |
|---|---|
| (1) Compound of the present invention | 20 parts by weight |
| (2) N,N-dimethylacetamide | 20 parts by weight |
| (3) Polyoxyethylene tristyryl phenyl ether | 10 parts by weight |
| (4) Calcium dodecylbenzene sulfonate | 2 parts by weight |
| (5) Xylene | 48 parts by weight |

The above components are uniformly mixed and dissolved to obtain an emulsifiable concentrate.

### Formulation Example 4

| | |
|---|---|
| (1) Clay | 68 parts by weight |
| (2) Sodium lignin sulfonate | 2 parts by weight |
| (3) Polyoxyethylenealkylaryl sulfate | 5 parts by weight |
| (4) White carbon | 25 parts by weight |

The mixture of the above components is mixed with compound of the present invention in a weight ratio of 4:1 to obtain a wettable powder.

### Formulation Example 5

| | | |
|---|---|---|
| (1) Compound of the present invention | 50 | parts by weight |
| (2) Sodium alkylnaphthalene sulfonate condensa- | 2 | parts by weight |
| tion product of formaldehyde | | |
| (3) Silicone oil | 0.2 | part by weight |
| (4) Water | 47.8 | parts by weight |
| (5) Sodium polycarboxylate | 5 | parts by weight |
| (6) Anhydrous sodium sulfate | 42.8 | parts by weight |

The above components are uniformly mixed and pulverized to obtain a base liquid, and are added, and the mixture is uniformly mixed, granulated and dried to obtain water-dispersible granules.

### Formulation Example 6

| | | |
|---|---|---|
| (1) Compound of the present invention | 5 | parts by weight |
| (2) Polyoxyethyleneoctylphenyl ether | 1 | part by weight |
| (3) Polyoxyethylene alkyl ether phosphoric | 0.1 | part by weight |
| acid ester | | |
| (4) Granular calcium carbonate | 93.9 | parts by weight |

The above components (1) to (3) are preliminarily uniformly mixed and diluted with a proper amount of acetone, and then the mixture is sprayed onto the component (4), and acetone is removed to obtain granules.

### Formulation Example 7

| | |
|---|---|
| (1) Compound of the present invention | 2.5 parts by weight |
| (2) N,N-dimethylacetamide | 2.5 parts by weight |
| (3) Soybean oil | 95.0 parts by weight |

The above components are uniformly mixed and dissolved to obtain an ultra low volume formulation.

### Formulation Example 8

| | | |
|---|---|---|
| (1) Compound of the present invention | 40 | parts by weight |
| (2) Potassium polyoxyethylene styryl phenyl | 4 | parts by weight |
| ether phosphate | | |
| (3) Silicone oil | 0.2 | part by weight |
| (4) Xanthan gum | 0.1 | part by weight |
| (5) Ethylene glycol | 5 | parts by weight |
| (6) Water | 50.7 | parts by weight |

The above components are uniformly mixed and pulverized to obtain a water-based suspension concentrate.

### Formulation Example 9

| | |
|---|---|
| (1) Compound of the present invention | 10 parts by weight |
| (2) Diethylene glycol monoethyl ether | 80 parts by weight |
| (3) Polyoxyethylenealkyl ether | 10 parts by weight |

The above components are uniformly mixed to obtain a soluble concentrate.

The entire disclosure of Japanese Patent Application No. 2008-292881 filed on Nov. 17, 2008 including specification, claims and summary is incorporated herein by reference in its entirety.

1. A pyridine derivative represented by the formula (I) or its salt: 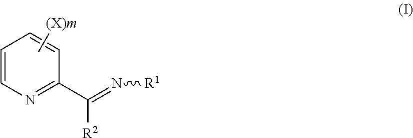
wherein R¹ is alkyl, cycloalkyl, alkoxyalkyl or OR³; R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, phenoxy which may be substituted by B, hydroxyl, NR⁴R⁵, OCOR⁶, OCOOR⁶, OS(O)ₙR⁶, aryl which may be substituted by B, heteroaryl which may be substituted by B, COR⁶, COOR⁶, S(O)ₙR⁶or CONR⁴R⁵; R³ is alkyl which may be substituted by D, cycloalkyl which may be substituted by E, alkenyl which may be substituted by D, alkynyl which may be substituted by D, phenylalkyl which may be substituted by E, pyridylalkyl which may be substituted by E, phenyl which may be substituted by E, silyl which is substituted by E, N-alkylcarbamoyl, N-alkoxycarbamoyl or N,N-dialkylcarbamoyl; R⁴ is a hydrogen atom or alkyl; R⁵ is a hydrogen atom, alkyl which may be substituted by A, cycloalkyl which may be substituted by B, arylalkyl which may be substituted by B, heteroarylalkyl which may be substituted by B, COR⁶, COOR⁶, S(O)ₙR⁶ or CH₂CN; R⁶ is alkyl, haloalkyl, or aryl which may be substituted by B; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; D is at least one substituent selected from the group consisting of cycloalkyl, halogen, alkoxy, haloalkoxy, alkylthio, cyano, nitro, alkylcarbonyl, haloalkylcarbonyl, alkoxycarbonyl and alkylsilyl; E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl, alkylsilyl, tetrahydropyranyl, 1,3-dioxolan-2-yl and N,N-dialkylamino; m is an integer of from 1 to 4; and n is 1 or 2.

2. A pyridine derivative represented by the formula (I) or its salt: 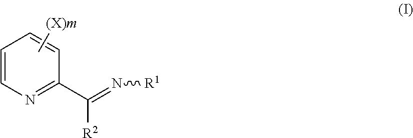
wherein R¹ is alkyl, cycloalkyl, alkoxyalkyl or OR³; R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, or phenoxy which may be substituted by B; R³ is alkyl which may be substituted by D, cycloalkyl which may be substituted by E, alkenyl which may be substituted by D, alkynyl which may be substituted by D, phenylalkyl which may be substituted by E, pyridylalkyl which may be substituted by E, phenyl which may be substituted by E, silyl which is substituted by E, N-alkylcarbamoyl, N-alkoxycarbamoyl or N,N-dialkylcarbamoyl; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; D is at least one substituent selected from the group consisting of cycloalkyl, halogen, alkoxy, haloalkoxy, alkylthio, cyano, nitro, alkylcarbonyl, haloalkylcarbonyl, alkoxycarbonyl and alkylsilyl; E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl, alkylsilyl, tetrahydropyranyl, 1,3-dioxolan-2-yl and N,N-dialkylamino; and m is an integer of from 1 to 4.

3. The pyridine derivative or its salt according to claim 2, wherein X is alkyl which may be substituted by A, cycloalkyl which may be substituted by B, halogen, nitro, cyano, or alkoxy which may be substituted by A; R³ is alkyl which may be substituted by D, cycloalkyl which may be substituted by E, or alkenyl which may be substituted by D; and E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl and alkylsilyl.

4. The pyridine derivative or its salt according to claim 3, wherein R¹ is OR³; and R² is 1H-1,2,4-triazol-1-yl, 1H-imidazol-1-yl, 1H-1,2,3-triazol-1-yl or 4H-1,2,4-triazol-4-yl.

5. A pesticide containing the pyridine derivative or its salt as defined in claim 1, as an active ingredient.

6. An agricultural and horticultural pesticide containing the pyridine derivative or its salt as defined in claim 1, as an active ingredient.

7. An insecticide, miticide, nematicide or soil pesticide containing the pyridine derivative or its salt as defined in claim 1, as an active ingredient.

8. An insecticide or miticide containing the pyridine derivative or its salt as defined in claim 1, as an active ingredient.

9. A method for controlling a pest, which comprises applying an effective amount of the pyridine derivative or its salt as defined in claim 1.

10. A process for producing a pyridine derivative represented by the formula (I) or its salt: 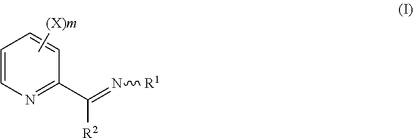
wherein R¹ is alkyl, cycloalkyl, alkoxyalkyl or OR³; R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, phenoxy which may be substituted by B, hydroxyl, NR⁴R⁵, OCOR⁶, OCOOR⁶, OS(O)ₙR⁶, aryl which may be substituted by B, heteroaryl which may be substituted by B, COR⁶, COOR⁶, S(O)ₙR⁶or CONR⁴R⁵; R³ is alkyl which may be substituted cycloalkyl which may be substituted by E, alkenyl which may be substituted by D, alkynyl which may be substituted by D, phenylalkyl which may be substituted by E, pyridylalkyl which may be substituted by E, phenyl which may be substituted by E, silyl which is substituted by E, N-alkylcarbamoyl, N-alkoxycarbamoyl or N,N-dialkylcarbamoyl; R⁴ is a hydrogen atom or alkyl; R⁵ is a hydrogen atom, alkyl which may be substituted by A, cycloalkyl which may be substituted by B, arylalkyl which may be substituted by B, heteroarylalkyl which may be substituted by B, COR⁶, COOR⁶, S(O)ₙR⁶ or CH₂CN; R⁶ is alkyl, haloalkyl, or aryl which may be substituted by B; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; D is at least one substituent selected from the group consisting of cycloalkyl, halogen, alkoxy, haloalkoxy, alkylthio, cyano, nitro, alkylcarbonyl, haloalkylcarbonyl, alkoxycarbonyl and alkylsilyl; E is at least one substituent selected from the group consisting of alkyl, haloalkyl, cycloalkyl, halogen, alkoxy, haloalkoxy, alkylthio, cyano, nitro, alkylcarbonyl, haloalkylcarbonyl, alkoxycarbonyl, alkylsilyl, tetrahydropyranyl, 1,3-dioxolan-2-yl and N,N-dialkylamino; m is an integer of from 1 to 4; and n is 1 or 2, which comprises
(1) reacting a compound represented by the formula (III): 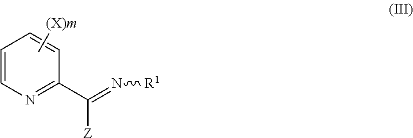
wherein Z is halogen; and R¹, X and m are as defined above, with a compound represented by the formula (IV): R²—H wherein R² is as defined above; or
(2) reacting a compound represented by the formula (VI): 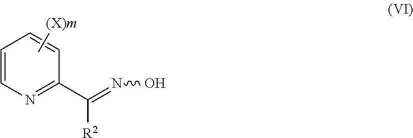
wherein R², X and m are as defined above, with a compound represented by the formula (VII): R³-L wherein L is halogen, alkylsulfonyloxy, trifluoromethanesulfonyloxy, or benzenesulfonyloxy which may be substituted by alkyl; and R³ is as defined above.

11. A compound represented by the formula (VI) or its salt: 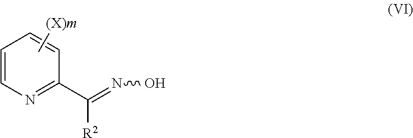
wherein R² is 1H-1,2,4-triazol-1-yl which may be substituted by alkyl, 1H-imidazol-1-yl which may be substituted by alkyl, 1H-1,2,3-triazol-1-yl which may be substituted by alkyl, or 4H-1,2,4-triazol-4-yl which may be substituted by alkyl; X is alkyl which may be substituted by A, cycloalkyl which may be substituted by B, halogen, nitro, cyano, alkoxy which may be substituted by A, cycloalkyloxy which may be substituted by B, arylalkoxy which may be substituted by B, silylalkyl which is substituted by B, silylalkoxy which is substituted by B, alkylthio which may be substituted by A, alkenyl which may be substituted by A, alkynyl which may be substituted by A, alkenyloxy which may be substituted by A, alkynyloxy which may be substituted by A, phenoxy which may be substituted by B, hydroxyl, NR⁴R⁵, OCOR⁶, OCOOR⁶, OS(O)ₙR⁶, aryl which may be substitute by B, heteroaryl which may be substituted by B, COR⁶, COOR⁶, S(O)ₙR⁶or CONR⁴R⁵; R⁴ is a hydrogen atom or alkyl; R⁵ is a hydrogen atom, alkyl which may be substituted by A, cycloalkyl which may be substituted by B, arylalkyl which may be substituted by B, heteroarylalkyl which may be substituted by B, COR⁶, COOR⁶, S(O)ₙR⁶ or CH₂CN; R⁶ is alkyl, haloalkyl, or aryl which may be substituted by B; A is at least one substituent selected from the group consisting of cycloalkyl, halogen, alkoxy and haloalkoxy; B is at least one substituent selected from the group consisting of alkyl, haloalkyl, cycloalkyl, halogen, alkoxy and haloalkoxy; m is an integer of from 1 to 4; and n is 1 or 2.
